(19) United States
(12) Patent Application Publication
(10) Pub. No.: US 2013/0184173 A1
(43) Pub. Date: Jul. 18, 2013

(54) BIOMARKERS FOR MULTIPLE SCLEROSIS

(71) Applicants: Amit Bar-Or, Westmount (CA); Elizabeth Colman, Bangor, ME (US); Katja Geling, Badersleben (DE)

(72) Inventors: Amit Bar-Or, Westmount (CA); David R. Colman, Westmount (CA); Dhaunchak Ajit Singh, Lasalle (CA)

(73) Assignee: The Royal Institution for the Advancement of Learning/Mcgill University, Montreal (CA)

(21) Appl. No.: 13/641,137

(22) PCT Filed: Apr. 14, 2011
(86) PCT No.: PCT/CA11/00425

§ 371 Date: Apr. 4, 2013

Related U.S. Application Data
(60) Provisional application No. 61/324,077, filed on Apr. 14, 2010.

(51) Int. Cl. G01N 33/68

(52) U.S. Cl. CPC G01N 33/6896
USPC 506/9; 435/24; 435/4; 435/23; 506/12; 435/7.92; 435/7.4

(57) ABSTRACT

Biomarkers that can be used for the diagnosis and prognosis of multiple sclerosis in pediatric patients presenting with clinically isolated syndrome or acquired demyelination syndrome are described.

### FIELD OF THE INVENTION

This invention relates to the identification of novel initiating targets and prognostic and diagnostic markers of multiple sclerosis in pediatric-onset CNS demyelination.

### BACKGROUND OF THE INVENTION

Multiple sclerosis (MS) is a chronic neurological disorder defined by recurrent episodes of central nervous system (CNS) demyelination, ultimately culminating in physical and cognitive disability. While it is rare in the pediatric population, MS in children is likely to have a profound impact on their lifetime academic, social, and vocational achievements. The National Multiple Sclerosis Society estimates that 2% to 5% of MS patients experience their first symptoms before age 18 years, and MS is the leading cause of neurological disability in young adults in the western hemisphere.

MS is considered to be an autoimmune disease of the CNS, in which the immune system repeatedly attacks the CNS, damaging both the myelin and axons. Since the site and severity of repeated immune attacks are different across individuals, the physical or cognitive disability caused is unpredictable and varies widely. Elucidating the earliest targets of the disease, and developing biomarkers that can predict the severity or progression of the disease, are of paramount importance.

Not everyone who has an initial episode of CNS inflammation or demyelination will go on to develop multiple sclerosis. When optic neuritis is the presenting symptom, roughly 50% of these patients will recover fully; the remainder will go on to develop MS over a number of years.

The varied clinical phenotypes of initial acute CNS demyelination, termed clinically isolated syndromes (CIS) or acquired demyelinating syndrome (ADS), include optic neuritis, transverse myelitis, hemisensory or hemi-motor syndromes, cerebellar or brainstem dysfunction, alone (mono-symptomatic CIS), in combination (polysymptomatic CIS), or associated with encephalopathy (acute disseminated encephalomyelitis, ADEM). Other clinical features that suggest demyelination of the brain, spinal cord, or optic nerves include numbness or weakness that is either unilateral or bilateral, dizziness, double vision, loss of vision, facial weakness or numbness, bladder problems, or a band-like sensation around the trunk that patients may mistake as stomach pain. Symptoms typically last longer than 24 hours; frequently from days to weeks.

Generally 50% or more of patients presenting with CIS/ADS will develop MS. However, the underlying cause, initial site of injury, and the factors influencing the transformation of the first CNS demyelination episode to multiple sclerosis (MS) are not well understood. Moreover, it can be difficult to distinguish between pediatric MS and other childhood CNS inflammatory demyelinating disorders such as ADEM. No single diagnostic test exists to confirm the presence of MS or to rule it out definitively. At a minimum, diagnosis requires a clinical episode consistent with CIS/ADS along with either a follow-up MRI study that shows new lesions or a second episode at least 30 days after the initial clinical event that involves a different region of the CNS. Over the years, studies of cerebrospinal fluid (CSF) from adult MS patients have identified abnormal elevations in levels of several compact myelin proteins, such as myelin basic protein (MBP), proteolipid protein (PLP1), NogoA and other inter-nodal proteins, and implicated these molecules as putative disease targets or potential biomarkers of disease activity. However, it remains unclear to what extent these molecules represent early/initiating targets, or reflect consequences of CNS injury.

Thus, the ability to predict whether a child with CIS/ADS will develop the recurrent demyelination that characterizes MS remains an ongoing challenge. Prompt diagnosis in children is hampered by a lack of clinical, epidemiological, or biological risk markers predictive of MS. At the same time, the advent of disease-modifying therapies for MS and the recent evidence of improved long-term outcome associated with early initiation of therapy, emphasize the need for prompt diagnosis for children.

In addition, children presenting with an initial acquired CNS demyelinating syndrome (ADS) provide an opportunity to study the earliest events in the MS spectrum. Only a portion of children presenting with ADS will subsequently develop further disease activity establishing the diagnosis of MS. The remaining children will not develop further disease activity, and hence represent a population with transient (monophasic) CNS-directed inflammation. Elucidating the earliest sites of disease in children presenting with ADS, and identifying biomarkers at time of ADS that can distinguish patients destined for recurrent disease activity (MS) from those with a monophasic illness, would represent major advances both in diagnostics and in understanding of mechanisms involved in MS initiation.

There is a need therefore for predictive and diagnostic markers of MS, as well as the identification of early targets of CNS injury in pediatric-onset demyelination.

### SUMMARY OF THE INVENTION

The identification of early/initiating molecular targets of disease in multiple sclerosis (MS), and of novel biomarkers that can be used to predict outcome from initial episodes of CNS inflammation and/or to diagnose MS, are provided herein.

In an aspect, there are provided herein methods for determining prognosis of a subject with CIS/ADS, comprising the steps of determining the level of a biomarker in a fluid sample taken from the subject and in samples taken from control subjects, wherein a change in concentration of the biomarker in the subject relative to concentration of the biomarker in control subjects indicates the subject is at high risk of developing multiple sclerosis.

There are also provided herein methods for diagnosing multiple sclerosis in a patient having pediatric-onset CNS demyelination, comprising the steps of determining the level of a biomarker in a fluid sample taken from the patient and in samples from control subjects, wherein a change in concentration of the biomarker in the patient relative to concentration of the biomarker in control subjects is diagnostic of multiple sclerosis.

In one aspect, the control subject is a subject who has CIS/ADS but does not develop MS. In other aspects, the control subject may be a subject who does not have CIS/ADS, a subject who does not have pediatric onset CNS demyelination, or a healthy subject.

In other embodiments, there are provided methods for monitoring disease progression in a subject having multiple sclerosis, comprising the steps of isolating fluid samples from a subject at different time points and monitoring the level of a biomarker in the fluid samples taken from the subject, wherein an increase or decrease of the concentration of the biomarker in the samples over time indicates progression or regression of multiple sclerosis.

In some embodiments, there are provided methods for monitoring therapeutic efficacy of an anti-MS treatment, comprising the steps of isolating fluid samples from a subject receiving anti-MS treatment at different time points and monitoring the level of a biomarker in the fluid samples taken from the subject, wherein an increase or decrease of the concentration of the biomarker in the samples over time indicates progression or regression of multiple sclerosis, such that the therapeutic efficacy of the anti-MS treatment is monitored.

In an aspect, the fluid samples may be cerebrospinal fluid (CSF), blood, lymph or serum, or any other suitable fluid or tissue sample.

It should be understood that for some biomarkers, elevation of the concentration of the biomarker in the subject indicates the subject is at high risk for developing multiple sclerosis (MS) or has MS, whereas for other markers, diminution of the concentration of the biomarker in the subject indicates the subject is at high risk for developing multiple sclerosis or has MS.

In an embodiment the biomarker is a nodal protein or a protein associated with the axoglial-apparatus in the CNS. In another embodiment, the biomarker is a protein associated with cell adhesion, extracellular matrix or immunological response. In other embodiments, the biomarker is any protein described herein, including proteins listed in Tables 3, 4 and/or 6, and/or proteins listed in FIGS. 6 and/or 8 which were detected in the CSF.

The subject may be a mammal, including a human. In a particular embodiment, the subject is a pediatric subject and/or a patient with pediatric-onset demyelination (CIS/ADS).

There are also provided herein kits for diagnosing or prognosing MS in a subject. Such kits may, for example, include at least one agent that binds at least one biomarker protein of the invention specifically, and instructions for use thereof. The agent may be, for example, an antibody specific for the biomarker protein or a mass spectrometry probe specific for the biomarker protein.

In other embodiments, at least two, three, four, five or six biomarker proteins may be used in the methods and kits of the invention.

In further embodiments, there are provided methods for predicting the likelihood of a subject with CIS/ADS developing multiple sclerosis (MS), wherein the level of a protein in a first fluid sample obtained from the subject is assayed, and then compared to the level of the protein in a second fluid sample obtained from a control subject, wherein a change in the level of the protein in the first fluid sample compared to the level of the protein in the second fluid sample indicates the likelihood of the subject developing MS. In one aspect, the control subject is a subject who has CIS/ADS but does not develop MS. In other aspects, the control subject may be a subject who does not have CIS/ADS, a subject who does not have pediatric onset CNS demyelination, or a healthy subject. The protein may be, for example, a nodal protein, a protein associated with the axoglial-apparatus in the CNS, and/or a protein associated with cell adhesion, extracellular matrix or immunological response. In other embodiments, the protein is one or more of the proteins listed in Tables 3, 4 and/or 6 and/or FIGS. 6 and/or 8. In yet other embodiments, more than one protein or a combination of proteins is used.

In a particular embodiment, the subject is a pediatric subject.

In other embodiments, the biomarker or protein used in the methods provided herein may be reelin, fibulin-1, fibulin-3, collagen alpha-1 (VI), carboxypeptidase E, or a combination thereof. In an aspect, elevation of the concentration of one or more of these biomarkers or proteins in the subject, e.g., in the CSF, compared to control indicates that the subject has or is at risk of developing multiple sclerosis, or that the subject has active CIS/ADS. In other embodiments, the biomarker or protein used in the methods provided herein may be brain acid soluble protein 1 (neuronal axonal membrane protein), chromagranin A, macrophage colony-stimulating factor 1 receptor, fructose-bisphosphate aldolase A, or a combination thereof. In an aspect, reduction of the concentration of the biomarker or protein in the subject, e.g., in the CSF, compared to control indicates that the subject has or is at risk of developing multiple sclerosis, or that the subject has active CIS/ADS.

In still other embodiments, the biomarker or protein used in the methods provided herein may be alpha-1-B-glycoprotein, platelet glycoprotein Ib, platelet p47 protein (pleckstrin), platelet basic protein (C-X-C motif chemokine 7), antithrombin III, apolipoprotein A-I, attractin, carboxypeptidase N, complement components C1r and C7, hepatocyte growth factor activator, or a combination thereof. In an aspect, elevation of the concentration of the biomarker or protein in the subject, e.g., in the serum, compared to control indicates that the subject has or is at risk of developing multiple sclerosis, or that the subject has active CIS/ADS. In yet other embodiments, the biomarker or protein used in the methods provided herein may be cytoplasmic actin 2, extracellular matrix protein 1, filamin A, neutrophil defensin 3, neutraphil gelatinase-associated lipocalin, SH3 domain-binding glutamic acid-rich-like protein 3, talin-1, thrombospondin-1, transgelin-2, tropomyosin 3, tropomyosin alpha-4 or a combination thereof. In an aspect, reduction of the concentration of the biomarker or protein in the subject, e.g., in the serum, compared to control indicates that the subject has or is at risk of developing multiple sclerosis, or that the subject has active CIS/ADS.

In a further embodiment, the biomarker or protein used in the methods of the invention comprises: a first biomarker or protein which is Reelin, PKC substrate 80K-H, CASPR4, corticosteroid binding globulin precursor, secreted frizzled-related protein 4, glutamate receptor AMPA 4 isoform 3, carboxypeptidase E preprotein, Tenascin R, or a combination thereof; and/or a second biomarker or protein which is CamKIIa, CD163 antigen isoform b, Tissue inhibitor of metalloproteinase 1, Growth associated protein 43 isoform 1, Sulfatase 2 isoform b precursor, Apolipoprotein C-II precursor, ADAM 22, Peptidylprolyl isomerase A, or a combination thereof; and elevation of the concentration of the first biomarker or protein in the CSF in the subject compared to control and/or reduction of the concentration of the second biomarker or protein in the CSF in the subject compared to control indicates that the subject has or is at risk of developing multiple sclerosis, or that the subject has active CIS/ADS.

### BRIEF DESCRIPTION OF THE DRAWINGS

Having thus generally described the nature of the invention, reference will now be made to the accompanying drawings, showing by way of illustration, an embodiment or embodiments thereof, and in which:

FIG. 1 shows the molecular function assignment of identified proteins, wherein

molecular classes that might be relevant for MS etiology are highlighted in bold. The molecular function assignment of identified proteins suggests perturbed axon-to-glia interaction in addition to immune system involvement in children with acquired demyelinating syndromes;

FIG. 2 illustrates the absence of compact myelin auto-antigens in the CSF, wherein the top shows a Ponseau S staining of the PVDF membrane prior to immunoblotting with myelin basic protein (MBP) and proteolipid protein (PLP1) antibodies respectively;

FIG. 3 shows elevated levels of axoglial-apparatus molecules in children that later develop MS, wherein the relative levels of OMgp and Gliomedin quantified from immunoblots using quantity one software from Biorad are shown. As expected the human IgG levels are also elevated in the active cohort whereas the levels of CSF resident transthyretin are unaltered;

FIG. 4 shows a few examples of the CSF components (for different proteins) for which the peptide profiling shows significant difference in the concentration between the two cohorts;

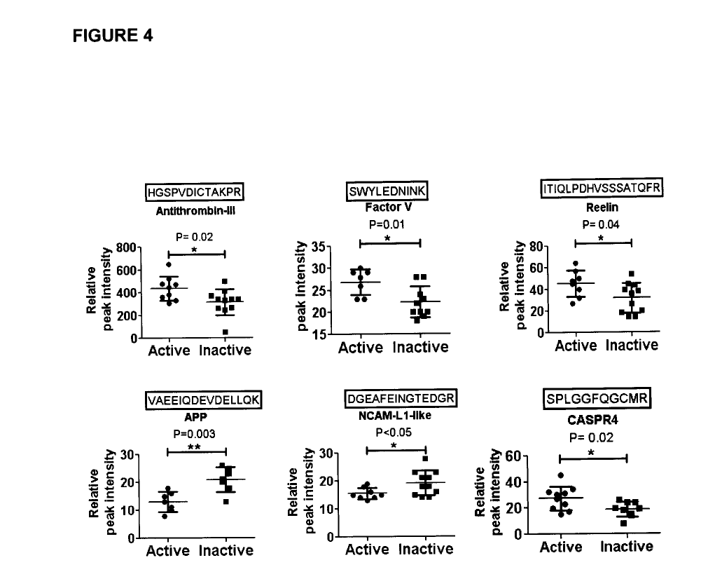

FIG. 5 shows a histogram of occurrences of the difference between observed and predicted retention index for approximately 20,000 peptides with Mascot identification scores above 40, taken from PPD Biomarker Discovery Science Inc.'s human plasma proteome library, wherein retention index is a standardized measure of chromatographic retention time, with about 17 RI units per minute, using a 1-hour gradient;

FIG. 6 shows axoglial apparatus proteins detected in pediatric ADS CSF, their expression in isolated fractions from human CNS, and a model of the human axoglial apparatus;

FIG. 7 shows absence of the compact myelin auto-antigens in the CSF;

FIG. 8 shows that the CSF proteome of children presenting with ADS is different between ADS-MS and ADS-Monophasic cohorts. In FIG. 8B, the mass spectroscopy data was validated using immunoblotting. Elevated GLDN, OMG and IgG levels were identified in patients that later experienced new disease activity, suggesting that axoglial-apparatus was damaged to larger extent; and

### DETAILED DESCRIPTION

We report herein the identification of early/initiating molecular targets of disease in multiple sclerosis (MS), and the identification of novel biomarkers that can be used to predict outcome from initial episodes of CNS inflammation and/or diagnose MS.

Cerebrospinal fluid samples collected from children during initial presentation of CNS-inflammation that may or may not subsequently be diagnosed as multiple sclerosis (MS), were subjected to a large-scale proteomics screen. Unexpectedly, major compact myelin membrane proteins typically implicated in MS were not detected. However, multiple molecules that localize to the node of Ranvier and the surrounding axoglial apparatus membrane were identified, and indicated perturbed axon-glial interactions in those children destined for diagnosis of MS.

To gain insight into the early events leading to an acute attack of CNS demyelination, and to search for putative prognostic markers of MS in pediatric-onset CNS demyelination, an unbiased non-gel based proteomics screen was performed on CSF samples obtained from 24 children during the first episode of CNS demyelination. Twenty of the samples were analyzed, of which, during a two year follow up, 8 children developed MS (classified as active) and 11 stayed inactive (1 sample was excluded from the study). Of the 19, 4 children were diagnosed with ADEM. Quite unexpectedly the auto-antigens that experimentally induce encephalomyelitis in animals including PLP1 and MBP were absent from the CSF. Instead, it is reported herein that several key molecules characteristic of the node of Ranvier and the paranodal region were readily detectable, suggesting structurally compromised nodes. Evidence is provided for injury to the node and surrounding regions that might lead to conduction block, which if prolonged, might target myelin in MS. It is also reported that the proteomic signature may be used as an accurate predictor of disease progression in MS.

In order to investigate potential early targets and biomarkers of MS, the large-scale non-gel based proteome study was performed on twenty matched CSF and serum samples obtained from children at the time of onset of CIS/ADS. Of these twenty children, 8 subsequently experienced new disease activity consistent with MS, whereas 11 remained inactive over a follow-up period of 3.0±1.0 years from initial ADS presentation; 1 sample was excluded from the study.

Of 1038 proteins identified, 788 were unique to the CSF. Surprisingly, while peptides from compact myelin proteins including MBP and PLP (among the most abundant myelin proteins) were not detected, multiple peptides were found defining several adhesion, structural, scaffolding and cytoskeletal molecules that all localize to nodal/para-nodal and axolemmal regions. Presence of molecules critical for node-of-Ranvier and paranodal-assembly were confirmed by Western blot in ADS CSF samples. Not surprisingly, levels of hIgG were higher in the “active” vs. “inactive” groups (p=0.026), and several nodal molecules were also differentially present in these groups, though only differences in Gliomedin (0.019) and OMGP (p=0.03) reached statistical significance in this small sample set.

Our data strongly suggest that the initial site of acute CNS injury in pediatric MS may be the region of the node-of-Ranvier, not the inter-node. We suggest that early injury to nodal/paranodal regions during acute CNS inflammation may impair impulse conduction by uncoupling glial support from the axon which may, in turn, contribute to transformation of ADS to MS.

As used herein, the terms “CIS”, “ADS”, “CIS/ADS” and “pediatric-onset CNS demyelination” are used interchangeably to refer to acute initial CNS inflammation or demyelination in pediatric subjects, which is known in the art as clinically isolated syndrome (CIS), acquired demyelinating syndrome (ADS) and/or pediatric-onset CNS demyelination.

We found numerous proteins showing clear statistically significant change in concentration in active vs. non-active patients. “Active” patients are children with initial ADS who subsequently exhibited further disease activity or were diagnosed with MS. “Inactive” or “non-active” patients are children with initial ADS who had no subsequent activity. Inactive ADS is also known in the art as monophasic ADS. The term “subject who has CIS/ADS but does not develop MS” is also used herein to refer to subjects with inactive or monophasic ADS.

Children were separated into active and inactive cohorts based on whether there was evidence of new disease activity subsequent to the initial episode. As used herein, disease “activity” refers to developing over time one or more new clinical attacks (e.g. of CNS demyelination or inflammation), or having evidence of new brain lesions developing over time as determined by magnetic resonance imaging (MRI). In general, active patients are at high risk of developing MS or have been diagnosed with MS. Many of the proteins which showed clear statistically significant change in concentration in active vs. non-active patients have implications in extracellular matrix regulation, cellular adhesion, and immunological response.

In the CSF, upward expressed proteins in the active cohort include CASPR4 and Tenascin R, which are known axoglial apparatus components. In addition to these two proteins, other upwardly expressed proteins include macrophage migration inhibitory factor, reelin, fibulin-1, fibulin-3 (EGF-containing fibulin-like extracellular matrix protein 1), collagen alpha-1 (VI), carboxypeptidase E, ionotrophic glutamate receptor (AMPA 4), junctional adhesion protein 2 (JAM-B) and protein kinase C substrate 80K-H isoform 2. Downward expressed proteins in the CSF in the active cohort include ADAM22 and olfactomedin-like 3, which are either known or potential axoglial-apparatus components. In addition, under-expression of several signalling molecules was found, such as tumor necrosis factor receptor superfamily member 21, retinol-binding protein 4, neuropilin 1, and calcium/calmodulin-dependent protein kinase II alpha.

In serum, upward expressed proteins in the active cohort include alpha-1-B-glycoprotein, platelet glycoprotein Ib, apolipoprotein A-I, attractin, carboxypeptidase N, several complement components of C1, C2 and C4, and complement H. Downward expressed proteins in serum in the active cohort include fibronectin 1, plasminogen, platelet p47 protein (pleckstrin), platelet basic protein (C-X-C motif chemokine 7), thrombospondin-1, transgelin-2, tropomyosin alpha-4 and vitamin D binding protein.

In one embodiment, the invention provides a method of diagnosing MS or prognosing MS (determining likelihood or risk of developing MS) in a test subject, which method comprises measuring at least one biomarker in a fluid sample from the test subject; and comparing the amount of the at least one biomarker in the fluid sample with the amount of biomarkers observed in a tissue sample from a control subject (e.g., a normal or healthy subject, a subject who has CIS/ADS but does not develop MS, etc.). The method further comprises comparing the measurements of at least two biomarkers in the fluid sample to a known profile of at least two biomarkers in a fluid sample from a control subject. A measurement indicating a 1.1-fold, or two-fold or greater increase (or decrease, depending on the biomarker) in a member of the first group of biomarkers, relative to normal or healthy fluid, is indicative of MS or a high risk of developing MS. The measuring typically comprises spectrometry or immunoassay. The spectrometry is typically mass spectrometry. A typical immunoassay would be an enzyme immunoassay, such as ELISA or a Western blot.

In one embodiment, the diagnostic and prognostic methods provided herein comprise use of an immunoassay, such as an ELISA type assay, that employs an antibody specific for a biomarker of the invention to detect the presence (or up-regulation or down-regulation) of the biomarker protein in a subject. Other assays include a radioimmune assay (RIA), a Western blot assay, or a slot blot assay. The immunoassay can be used either as a simple detection method, or to measure the amount or level of the biomarker present. Such measurements can be quantitative, by comparing measured amounts to a known or control amount of the biomarker, or can be used to compare relative amounts between different samples or subjects. The antibody can be immobilized on a substrate. The amount or presence of biomarker is typically determined via assay directed to a detectable marker or other indication of binding of the antibody to the biomarker. Measures can be based on, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Methods for performing these assays are readily known in the art.

Other methods can be used to detect the presence or amount of a biomarker in a sample. Typically, the biomarker is first captured on a substrate. Examples of such methods include, but are not limited to, gas phase ion spectrometry methods, optical methods, electrochemical methods, atomic force microscopy and radio frequency methods. In one embodiment, the method comprises mass spectrometry, such as “surface-enhanced laser desorption/ionization” or “SELDI”. SELDI refers to a method of desorption/ionization gas phase ion spectrometry (e.g., mass spectrometry) in which the analyte is captured on the surface of a SELDI probe that engages the probe interface. In “SELDI-MS,” the gas phase ion spectrometer is a mass spectrometer.

Those skilled in the art will appreciate additional variations suitable for the method of diagnosing or prognosing multiple sclerosis in a fluid sample from a subject through measurement or detection of a biomarker protein. These methods can also be used to monitor biomarker protein levels in fluid samples of a patient undergoing treatment for multiple sclerosis. The suitability of a multiple sclerosis-targeted therapeutic regimen for initial or continued treatment can be determined by monitoring biomarker protein levels using the methods provided herein.

The fluid sample can comprise cerebrospinal fluid (CSF), serum, blood, plasma, lymph or other suitable tissue specimen.

Kits for use in the diagnostic and prognostic applications described herein are also provided. Such kits can comprise a carrier, package or container that is compartmentalized to receive one or more containers such as vials, tubes, and the like, each of the container(s) comprising one of the separate elements to be used in the method. For example, the container(s) can comprise a probe that is or can be detectably labeled. The probe can be, for example, an antibody specific for a biomarker protein of the invention. Alternatively, the kit can comprise a mass spectrometry (MS) probe. The kit can also include a container comprising a reporter-means, such as a biotin-binding protein, e.g., avidin or streptavidin, bound to a detectable label, e.g., an enzymatic, florescent, or radioisotope label. The kit can include all or part of the amino acid sequence of the biomarker protein, or a nucleic acid molecule that encodes such amino acid sequences.

The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific application. Directions and or other information can also be included on an insert which is included with the kit.

In one embodiment, the invention provides a kit comprising at least one agent that binds a biomarker protein of the invention; and instructions for use of the at least one agent for determining status of MS in a subject. The kit can further comprise a container for housing the at least one agent. In some embodiments, the kit further comprises a substrate to which the at least one agent is bound. The agent can be an antibody that specifically binds the biomarker, and/or a mass spectrometry probe.

In one embodiment, the biomarker proteins of the invention include reelin, fibulin-1, fibulin-3 (EGF-containing fibulin-like extracellular matrix protein 1), collagen alpha-1 (VI) and/or carboxypeptidase E, for which the concentration is elevated in the CSF in subjects having or at risk of developing MS, or brain acid soluble protein 1 (neuronal axonal membrane protein), chromagranin A, macrophage colony-stimulating factor 1 receptor, and/or fructose-bisphosphate aldolase A, for which the concentration is reduced in the CSF in subjects having or at risk of developing MS.

In other embodiments, the biomarker proteins of the invention include alpha-1-B-glycoprotein, platelet glycoprotein Ib, platelet p47 protein (pleckstrin), platelet basic protein (C-X-C motif chemokine 7), antithrombin III, apolipoprotein A-I, attractin, carboxypeptidase N, complement components C1r and C7, and/or hepatocyte growth factor activator, for which the concentration is elevated in the serum in subjects having or at risk of developing MS, or cytoplasmic actin 2, extracellular matrix protein 1, filamin A, neutrophil defensin 3 and neutraphil gelatinase-associated lipocalin, SH3 domain-binding glutamic acid-rich-like protein 3, talin-1, thrombospondin-1, transgelin-2, tropomyosin 3 and/or tropomyosin alpha-4, for which the concentration is reduced in the serum in subjects having or at risk of developing MS.

In other embodiments, the biomarker proteins of the invention include nodal proteins or proteins associated with the axoglial-apparatus in the CNS. Non-limiting examples of such proteins include gliomedin and OMgp, for which the concentration is elevated in the CSF in subjects having or at risk of developing MS. In further embodiments, the biomarker proteins of the invention are proteins associated with extracellular matrix regulation, cellular adhesion, or immunological response.

In some embodiments, the biomarker proteins of the invention include the proteins listed in Tables 1-6 herein. It should be understood that any of the proteins described herein, or any combination of the proteins described herein, are contemplated for use as biomarker proteins of the invention.

The methods and kits of the invention may be based on detection and measurement of at least one, at least two, at least three, at least four, at least five or at least six of the biomarker proteins described herein. They may use only proteins for which elevated or reduced concentration indicates risk of MS, or they may use a combination of proteins, some of which increase in concentration and some of which decrease in concentration.

It is expected that the biomarkers, methods and kits of the invention will be useful for the diagnosis, prognosis and monitoring of other types of MS in addition to those described herein, such as adult-onset MS. It is also contemplated that the biomarkers, methods and kits of the invention may be useful for the diagnosis, prognosis and monitoring of other myelin diseases, in addition to MS, such as leukodystrophies (for which there are 36 subtypes).

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1

### Detection of Nodal Proteins and not Compact Myelin Proteins in the CSF

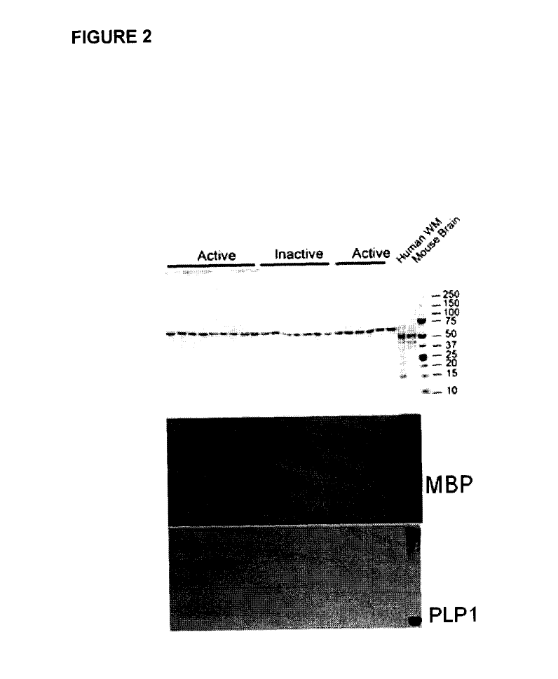

Injury to the CNS compact myelin is often associated with auto-antibodies to compact myelin proteins (proteolipid protein (PLP), myelin basic protein (MBP), 2′,3′-cyclic nucleotide 3′-phosphodiesterase (CNP)) in MS patients. The reason for synthesis and secretion of auto-antibodies directed against several myelin antigens in serum and CSF of MS patients remains unknown. In order to investigate whether these auto-antibodies are synthesized and secreted as a result of injury to the myelin sheath, resulting in CSF leakage of antigens, immunoblotting was used to look for myelin antigen in CSF obtained from children during the first episode of CNS demyelination. In humans, PLP1 and MBP together constitute about 75-90% of myelin protein content. However, no sign of PLP1, MBP or CNPase leakage into the CSF samples was identified (FIG. 2, wherein antibodies for two independent non-overlapping epitopes were used, and data not shown). No sign of PLP1, MBP or CNP leakage into the CSF was detected with mass spectroscopy either (data not shown).

To confirm the absence of myelin auto-antigens in the CSF samples and to identify novel biomarkers and auto-antigens in the CSF, a large scale, unbiased proteomic screen was performed on CSF obtained from 19 children during the first episode of CNS demyelination (24 samples were taken, and results were obtained from 19; see below). This screen confirmed the absence of the three most abundant myelin auto-antigens (PLP1, MBP and CNP) in the CSF of all the children tested, suggesting that myelin is not a primary target at this early stage.

### Example 2

### Proteomic Analysis

In order to identify biological protein markers in biofluids differentiating active (at high risk of developing MS or having MS) vs. inactive (at low risk) children presenting with clinically isolated syndrome (CIS) or acquired demyelination syndrome (ADS), proteomic analysis of biofluids from 24 children presenting with CIS/ADS (of which 19 samples were analyzed) was performed. A label-free differential proteomic analysis from PPD Biomarker Discovery Sciences (Menlo Park, Calif.) was performed using liquid chromatography-mass spectrometry (LC-MS). A one-dimensional chromatography cerebral spinal fluid (CSF) and two-dimensional chromatography serum proteomic analysis were performed. For each of these biofluids, 14 abundant proteins were depleted by an antibody column prior to digestion by trypsin. Hundreds of proteins were tracked in each of the two analyses.

CSF and serum samples were collected from children participating in the Canadian prospective study of pediatric CNS inflammation. Children were followed from the time of an initial CNS inflammatory episode. The available follow-up enabled distinction between children who subsequently developed MS (those with active ADS), versus those who remained with single episodes of inflammation. Samples obtained from these children at the time of their initial presentation, were subjected to proteomics analysis using liquid chromatography-tandem mass spectroscopy (LC-MS/MS), as described herein.

CSF and serum samples were taken and children were assigned to each of two cohorts, either active or inactive. “Active” patients are children with initial ADS who subsequently exhibited further disease activity or were diagnosed with MS. “Inactive” or “non-active” patients are children with initial ADS who had no subsequent activity. Inactive ADS is also known in the art as monophasic ADS. Children were separated into active and inactive cohorts based on whether there was evidence of new disease activity subsequent to the initial episode. As used herein, disease “activity” refers to developing over time one or more new clinical attacks (e.g. of CNS demyelination or inflammation), or having evidence of new brain lesions developing over time as determined by magnetic resonance imaging (MRI). In general, active patients are either at high risk of developing MS or have been diagnosed with MS. It is noted that the modest cohort size limits the findings to relatively large effects.

PPD Biomarker Discovery Science Inc.'s proprietary label-free differential proteomic analysis was performed using liquid chromatography-mass spectrometry (LC-MS). One-dimensional chromatography (CSF) and two-dimensional chromatography (serum) proteomic analysis were performed. For each of these biofluids, 14 abundant proteins were depleted by an antibody column prior to digestion by trypsin. Hundreds of proteins were tracked in each of the two analyses.

A total of 16,190 molecular components were tracked and quantified for reporting in the one-dimensional CSF proteomic analysis. Median coefficients of variance (CV) for each cohort were between 33% to 30%. A summary of the number of components at several p-value ranges and CVs is shown in Table 1. A total of 7,367 components were linked to 3,839 peptide sequences, corresponding to 1,038 proteins with an identification threshold probability of 0.7, although many of these proteins are tracked as a “singleton” (only one peptide per protein) which represents a lower degree of identification confidence. There were 562 proteins with two or more peptides reporting. Although there were not many components at low p-value relative to the number of components tracked, the consistency between peptides (components) of certain specific proteins reveals that there were clear concentration changes of numerous specific proteins (Tables 3 and 6). This comment applies also to the serum analysis.

TABLE 1
The number of components having a p-value below the listed cutoffs, for
the profiling study of CSF, pair-wised comparison. A total of 16,190 components
were tracked and quantified in this 1D proteomic analysis.

| | | | | | Numerator (Active) | | Numerator (Inactive) | |
|---|---|---|---|---|---|---|---|---|
| Comparisons | P < 0.001 | P < 0.005 | P < 0.01 | P < 0.05 | Med CV | Avg CV | Med CV | Avg CV |
| C2-C1@D1 | 4 | 35 | 74 | 561 | 31% | 36% | 32% | 37% |

Cohorts labels: C1 = Active, C2 = Inactive, and D1 represents the single blood draw for the study.

For the two-dimension serum proteomic analysis, a total of 42,476 molecular components were tracked and quantified. Median coefficients of variance (CV) for each cohort were between 25% to 23%. A summary of the number of components at several p-value ranges and CVs is shown in Table 2. A total of 13,459 components were linked to 5,373 peptide sequences, corresponding to 1,329 proteins with an identification threshold probability of 0.7, although many of these proteins are tracked as a “singleton” (only one peptide per protein) which represents a lower degree of identification confidence. There were 279 proteins with two or more peptides reporting. Again, there were clear concentration changes of numerous specific proteins (Table 4).

TABLE 2
The number of components having a p-value below the listed cutoffs, for the
profiling study of serum, pair-wised comparison. A total of 42,476 components
were tracked and quantified in this 2D four-fraction proteomic analysis.

| | | | | | Numerator (Active) | | Numerator (Inactive) | |
|---|---|---|---|---|---|---|---|---|
| Comparisons | P < 0.001 | P < 0.005 | P < 0.01 | P < 0.05 | Med CV | Avg CV | Med CV | Avg CV |
| C2-C1@D1 | 6 | 73 | 191 | 1420 | 25% | 29% | 25% | 30% |

Cohorts labels: C1 = Active, C2 = Inactive, and D1 represents the single blood draw for the study.

While there was not an overwhelming difference between the two cohorts in terms of their biofluid compositions, there were numerous proteins which showed clear statistically significant change in concentration in the two cohorts. Many of these proteins have implications in extracellular matrix regulation, cellular adhesion, and immunological response.

In the CSF, upward expressed proteins in the active cohort include CASPR4 and Tenascin R, which are known axoglial apparatus components. In addition to these two proteins, other upwardly expressed proteins include macrophage migration inhibitory factor, reelin, fibulin-1, fibulin-3 (EGF-containing fibulin-like extracellular matrix protein 1), collagen alpha-1 (VI), carboxypeptidase E, ionotrophic glutamate receptor (AMPA 4), junctional adhesion protein 2 (JAM-B) and protein kinase C substrate 80K-H isoform 2. Downward expressed proteins in the CSF in the active cohort include ADAM22 and olfactomedin-like 3, which are either known or potential axoglial-apparatus components. In addition, under-expression of several signalling molecules was also found, such as tumor necrosis factor receptor superfamily member 21, retinol-binding protein 4, neuropilin 1, and calcium/calmodulin-dependent protein kinase II alpha.

In serum, upward expressed proteins in the active cohort include alpha-1-B-glycoprotein, platelet glycoprotein Ib, apolipoprotein A-I, attractin, carboxypeptidase N, several complement components of C1, C2 and C4, and complement H. Downward expressed proteins in serum in the active cohort include fibronectin 1, plasminogen, platelet p47 protein (pleckstrin), platelet basic protein (C-X-C motif chemokine 7), thrombospondin-1, transgelin-2, tropomyosin alpha-4 and vitamin D binding protein.

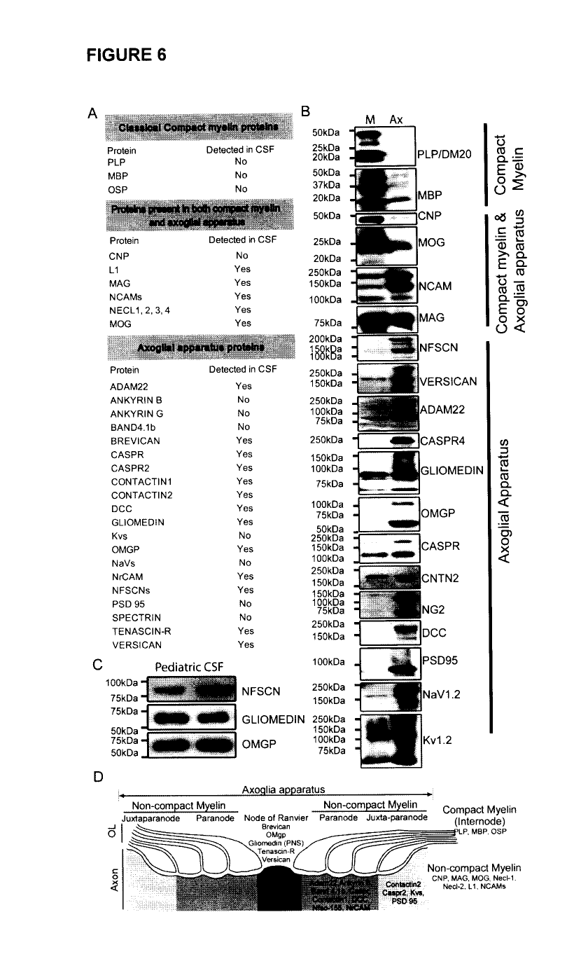

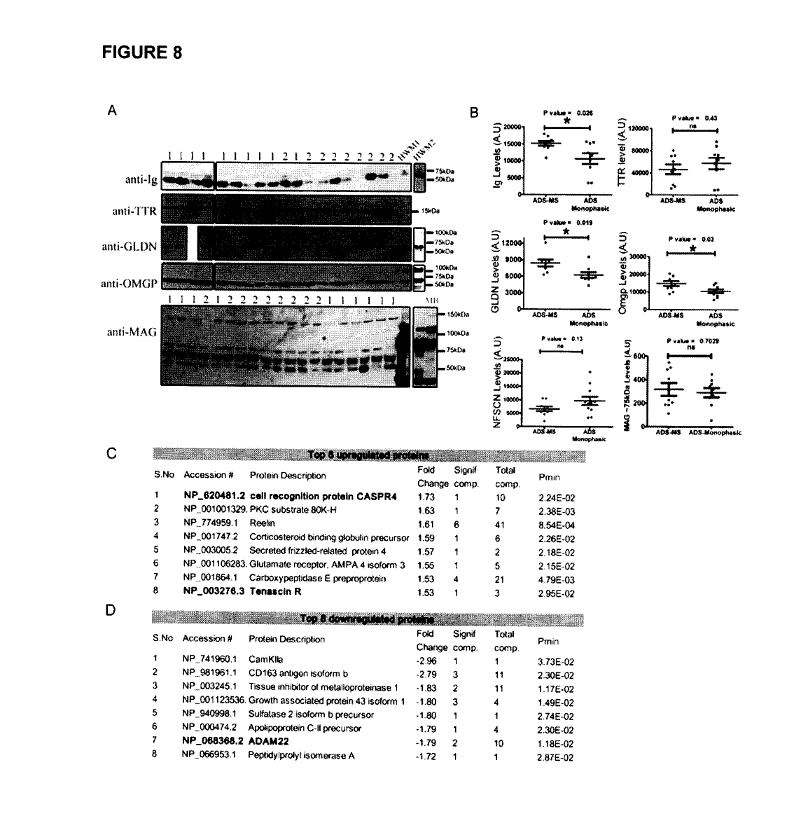

A condensed list of proteins showing statistically significant change in concentration for the two-group comparisons is provided in Tables 3 and 6 for CSF and Table 4 for serum. The lists in tables 3, 4 and 6 and FIGS. 6 and 8 are not intended to be complete but exemplify and summarize much of the primary results of the study. The lists are based on review of a combination of p-value, fold-change and relative identification confidence. Singletons are generally excluded even though there are many interesting components in that category. The Entrez gene ID number has been added (when provided by NCBI), along with information on the median fold-change and statistical effect size.

TABLE 3
Examples of proteins showing statistically significant concentration difference
between two cohorts (Active vs. Inactive) in the CSF 1D proteomic study.

| | | | | Med Exp | Fold | Effect | Trend |
|---|---|---|---|---|---|---|---|
| Accession # | gi # | Protein Description | Trend | Ratio | Change | Size | seq |
| NP_612457.1 | 20149322 | acylphosphatase 2 [Homo | DOWN | 0.70 | −1.43 | −1.02 | 1 |
| | | sapiens] | | | | | |
| NP_068368.2 | 21536386 | ADAM metallopeptidase | DOWN | 0.56 | −1.79 | −0.87 | 1 |
| | | domain 22 isoform 2 | | | | | |
| | | preproprotein [Homo | | | | | |
| | | sapiens] | | | | | |
| NP_569711.2 | 206597445 | alpha 1 type XVIII collagen | DOWN | 0.82 | −1.23 | −0.96 | 2 |
| | | isoform 3 precursor [Homo | | | | | |
| | | sapiens] | | | | | |
| NP_476508.2 | 55743106 | alpha 3 type VI collagen | DOWN | 0.72 | −1.38 | −1.09 | 1 |
| | | isoform 5 precursor [Homo | | | | | |
| | | sapiens] | | | | | |
| NP_001135749.1 | 214010183 | amyloid beta (A4) precursor- | DOWN | 0.64 | −1.55 | −1.37 | 1 |
| | | like protein 2 isoform 3 | | | | | |
| | | [Homo sapiens] | | | | | |
| NP_000474.2 | 32130518 | apolipoprotein C-II | DOWN | 0.56 | −1.79 | −0.91 | 1 |
| | | precursor [Homo sapiens] | | | | | |
| NP_002070.1 | 4504067 | aspartate | DOWN | 0.80 | −1.25 | −0.96 | 4 |
| | | aminotransferase 1 | | | | | |
| | | [Homo sapiens] | | | | | |
| NP_000051.1 | 4557373 | biotinidase precursor | UP | 1.23 | 1.23 | 1.10 | 5 |
| | | [Homo sapiens] | | | | | |
| NP_006308.3 | 30795231 | brain abundant, | DOWN | 0.61 | −1.63 | −0.95 | 7 |
| | | membrane attached | | | | | |
| | | signal protein 1 [Homo | | | | | |
| | | sapiens] | | | | | |
| NP_741960.1 | 25952118 | calcium/calmodulin- | DOWN | 0.34 | −2.96 | −1.40 | 1 |
| | | dependent protein kinase | | | | | |
| | | II alpha isoform 2 [Homo | | | | | |
| | | sapiens] | | | | | |
| NP_001864.1 | 4503009 | carboxypeptidase E | UP | 1.53 | 1.53 | 1.50 | 4 |
| | | preproprotein [Homo | | | | | |
| | | sapiens] | | | | | |
| NP_683880.1 | 23110957 | cathepsin H isoform b | UP | 1.48 | 1.48 | 1.27 | 1 |
| | | precursor [Homo sapiens] | | | | | |
| NP_001327.2 | 22538442 | cathepsin Z preproprotein | DOWN | 0.72 | −1.38 | −1.12 | 1 |
| | | [Homo sapiens] | | | | | |
| NP_981961.1 | 44889963 | CD163 antigen isoform b | DOWN | 0.36 | −2.79 | −1.47 | 3 |
| | | [Homo sapiens] | | | | | |
| NP_001001391.1 | 48255941 | CD44 antigen isoform 4 | UP | 1.22 | 1.22 | 1.22 | 1 |
| | | precursor [Homo sapiens] | | | | | |
| NP_620481.2 | 148664242 | cell recognition protein | UP | 1.73 | 1.73 | 1.71 | 1 |
| | | CASPR4 isoform 2 | | | | | |
| | | [Homo sapiens] | | | | | |
| NP_001839.2 | 87196339 | collagen, type VI, alpha 1 | UP | 1.33 | 1.33 | 1.22 | 8 |
| | | precursor [Homo sapiens] | | | | | |

| | Signif | Trend | Total | | | <ID |
|---|---|---|---|---|---|---|
| Accession # | comp. | comp. | comp. | Summary | Pmin | Prob.> |
| NP_612457.1 | 1 | 1 | 2 | 1(1)/2 | 4.67E−02 | −8.50 |
| NP_068368.2 | 2 | 2 | 10 | 2(2)/10 | 1.18E−02 | −0.23 |
| NP_569711.2 | 2 | 2 | 11 | 2(2)/11 | 4.85E−02 | −5.01 |
| NP_476508.2 | 1 | 1 | 9 | 1(1)/9 | 2.16E−02 | −1.45 |
| NP_001135749.1 | 1 | 1 | 1 | 1(1)/1 | 1.73E−02 | −4.68 |
| NP_000474.2 | 1 | 1 | 4 | 1(1)/4 | 2.30E−02 | −2.70 |
| NP_002070.1 | 4 | 4 | 18 | 4(4)/18 | 2.72E−02 | −6.24 |
| NP_000051.1 | 5 | 5 | 13 | 5(5)/13 | 1.26E−02 | −8.76 |
| NP_006308.3 | 8 | 8 | 10 | 8(8)/10 | 2.58E−03 | −5.39 |
| NP_741960.1 | 1 | 1 | 1 | 1(1)/1 | 3.73E−02 | −2.47 |
| NP_001864.1 | 4 | 4 | 21 | 4(4)/21 | 4.79E−03 | −9.70 |
| NP_683880.1 | 1 | 1 | 5 | 1(1)/5 | 1.81E−02 | −4.60 |
| NP_001327.2 | 1 | 1 | 3 | 1(1)/3 | 2.37E−02 | −4.93 |
| NP_981961.1 | 3 | 3 | 11 | 3(3)/11 | 2.30E−02 | −2.92 |
| NP_001001391.1 | 1 | 1 | 6 | 1(1)/6 | 2.93E−02 | −1.51 |
| NP_620481.2 | 1 | 1 | 10 | 1(1)/10 | 2.24E−02 | −6.55 |
| NP_001839.2 | 9 | 9 | 20 | 9(9)/20 | 1.55E−03 | −5.15 |

TABLE 4
Examples of proteins showing statistically significant concentration difference
between two cohorts (Active vs. Inactive) in the serum 2D proteomic study.

| | | | | Med Exp | Fold | Effect | Trend |
|---|---|---|---|---|---|---|---|
| Accession # | gi # | Protein Description | Trend | Ratio | Change | Size | seq |
| NP_003237.2 | 40317626 | thrombospondin 1 | DOWN | 0.66 | −1.51 | −1.34 | 32 |
| | | precursor [Homo sapiens] | | | | | |
| NP_002334.2 | 54607120 | lactotransferrin precursor | DOWN | 0.47 | −2.15 | −1.22 | 12 |
| | | [Homo sapiens] | | | | | |
| NP_001094077.1 | 154759291 | serine (or cysteine) | UP | 1.23 | 1.23 | 1.29 | 9 |
| | | proteinase inhibitor, clade A | | | | | |
| | | (alpha-1 antiproteinase, | | | | | |
| | | antitrypsin), member 10 | | | | | |
| | | [Homo sapiens] | | | | | |
| NP_003882.1 | 4506121 | protein Z, vitamin K- | UP | 1.38 | 1.38 | 1.17 | 5 |
| | | dependent plasma | | | | | |
| | | glycoprotein [Homo | | | | | |
| | | sapiens] | | | | | |
| NP_003555.1 | 4507357 | transgelin 2 [Homo sapiens] | DOWN | 0.58 | −1.73 | −1.10 | 5 |
| NP_000119.1 | 4503627 | platelet coagulation factor | DOWN | 0.78 | −1.28 | −0.97 | 4 |
| | | XI precursor [Homo | | | | | |
| | | sapiens] | | | | | |
| NP_000031.1 | 4557323 | apolipoprotein C-III | DOWN | 0.63 | −1.59 | −0.64 | 3 |
| | | precursor [Homo sapiens] | | | | | |
| NP_005150.1 | 4885049 | cardiac muscle alpha actin | DOWN | 0.66 | −1.51 | −1.15 | 3 |
| | | 1 proprotein [Homo | | | | | |
| | | sapiens] | | | | | |
| NP_003109.1 | 4507171 | secreted protein, acidic, | DOWN | 0.84 | −1.19 | −1.22 | 3 |
| | | cysteine-rich [Homo | | | | | |
| | | sapiens] | | | | | |
| NP_0000230.1 | 4557894 | lysozyme precursor [Homo | DOWN | 0.66 | −1.51 | −1.43 | 3 |
| | | sapiens] | | | | | |
| NP_0000164.4 | 150417978 | platelet glycoprotein Ib | DOWN | 0.82 | −1.21 | −1.09 | 2 |
| | | alpha polypeptide precursor | | | | | |
| | | [Homo sapiens] | | | | | |
| NP_0005555.2 | 38455402 | lipocalin 2 [Homo sapiens] | DOWN | 0.46 | −2.17 | −1.23 | 2 |
| NP_0005013.1 | 4826898 | profilin 1 [Homo sapiens] | DOWN | 0.61 | −1.65 | −1.04 | 2 |
| NP_0002695.1 | 4505981 | pro-platelet basic protein | DOWN | 0.76 | −1.32 | −1.19 | 2 |
| | | precursor [Homo sapiens] | | | | | |
| NP_0001104026.1 | 160420317 | filamin A, alpha isoform 2 | DOWN | 0.63 | −1.58 | −1.00 | 2 |
| | | [Homo sapiens] | | | | | |
| NP_0002655.2 | 156616273 | pleckstrin [Homo sapiens] | DOWN | 0.68 | −1.47 | −1.73 | 2 |
| NP_0000933.1 | 4758950 | peptidylprolyl isomerase B | DOWN | 0.69 | −1.45 | −1.27 | 2 |
| | | precursor [Homo sapiens] | | | | | |
| NP_0001129174.1 | 208973244 | tyrosine 3/tryptophan 5 - | DOWN | 0.62 | −1.62 | −0.49 | 1 |
| | | monooxygenase activation | | | | | |
| | | protein, zeta polypeptide | | | | | |
| | | [Homo sapiens] | | | | | |
| NP_0001034438.1 | 86788132 | EGF-containing fibulin-like | DOWN | 0.74 | −1.35 | −1.22 | 1 |
| | | extracellular matrix protein | | | | | |
| | | 1 precursor [Homo sapiens] | | | | | |
| NP_0004985.2 | 74272287 | matrix metalloproteinase 9 | DOWN | 0.85 | −1.18 | −1.07 | 1 |
| | | preproprotein [Homo | | | | | |
| | | sapiens] | | | | | |
| NP_0000588.2 | 55925576 | insulin-like growth factor | DOWN | 0.66 | −1.52 | −1.26 | 1 |
| | | binding protein 2, 36 kDa | | | | | |
| | | [Homo sapiens] | | | | | |
| NP_000558.2 | 55770842 | C-reactive protein, | UP | 1.22 | 1.22 | 1.28 | 1 |
| | | pentraxin-related [Homo | | | | | |
| | | sapiens] | | | | | |
| NP_004336.2 | 39753970 | cathelicidin antimicrobial | DOWN | 0.41 | −2.43 | −1.19 | 1 |
| | | peptide [Homo sapiens] | | | | | |
| NP_005208.1 | 4885179 | defensin, alpha 3 | DOWN | 0.51 | −1.96 | −1.11 | 1 |
| | | preproprotein [Homo | | | | | |
| | | sapiens] | | | | | |
| NP_002956.1 | 4506773 | S100 calcium-binding | UP | 1.29 | 1.29 | 1.78 | 1 |
| | | protein A9 [Homo sapiens] | | | | | |
| NP_001138632.1 | 223555975 | tropomyosin 4 isoform 1 | DOWN | 0.57 | −1.77 | −1.33 | 1 |
| | | [Homo sapiens] | | | | | |
| NP_003290.1 | 4507677 | heat shock protein 90 kDa | DOWN | 0.75 | −1.33 | 2.56 | 1 |
| | | beta, member 1 [Homo | | | | | |
| | | sapiens] | | | | | |
| NP_057381.3 | 166795301 | prenylcysteine oxidase 1 | DOWN | 0.78 | −1.29 | −0.93 | 1 |
| | | [Homo sapiens] | | | | | |
| NP_001919.2 | 42544239 | complement factor D | DOWN | 0.79 | −1.26 | −1.01 | 1 |
| | | preproprotein [Homo | | | | | |
| | | sapiens] | | | | | |
| NP_002635.2 | 31377806 | polymeric immunoglobulin | DOWN | 0.74 | −1.36 | −1.18 | 1 |
| | | receptor [Homo sapiens] | | | | | |
| NP_002584.2 | 157653329 | procollagen C- | DOWN | 0.71 | −1.41 | −1.16 | 1 |
| | | endopeptidase enhancer | | | | | |
| | | [Homo sapiens] | | | | | |
| NP_055140.1 | 7656991 | coronin, actin binding | DOWN | 0.63 | −1.59 | −1.32 | 1 |
| | | protein, 1C isoform 1 | | | | | |
| | | [Homo sapiens] | | | | | |
| P00748.2 | 119763 | RecName: | DOWN | 0.74 | −1.35 | −1.36 | 1 |
| | | Full = Coagulation factor XII; | | | | | |
| | | AltName: Full = Hageman | | | | | |
| | | factor; Short = HAF; | | | | | |
| | | Contains: RecName: | | | | | |
| | | Full = Coagulation factor XIIa | | | | | |
| | | heavy chain; Contains: | | | | | |
| | | RecName: Full = Beta-factor | | | | | |
| | | XIIa part 1; Contains: | | | | | |
| | | RecName: Full = Beta-factor | | | | | |
| | | XIIa part 2; Contains: | | | | | |
| | | RecName | | | | | |

| | Signif | Trend | Total | | | <ID |
|---|---|---|---|---|---|---|
| Accession # | comp. | comp. | comp. | Summary | Pmin | Prob.> |
| NP_003237.2 | 48 | 48 | 81 | 48(48)/81 | 3.54E−03 | −5.04 |
| NP_002334.2 | 12 | 12 | 37 | 12(12)/37 | 1.08E−02 | −2.19 |
| NP_001094077.1 | 14 | 14 | 31 | 14(14)/31 | 2099E−03 | −5.24 |
| NP_003882.1 | 5 | 5 | 16 | 5(5)/16 | 1.12E−02 | −5.42 |
| NP_003555.1 | 7 | 7 | 16 | 7(7)/16 | 1.52E−02 | −5.82 |
| NP_000119.1 | 4 | 4 | 37 | 4(4)/37 | 1.28E−02 | −5.19 |
| NP_000031.1 | 5 | 5 | 18 | 5(5)/18 | 3.05E−02 | −9.26 |
| NP_005150.1 | 3 | 3 | 26 | 3(3)/26 | 3.67E−02 | −6.68 |
| NP_003109.1 | 5 | 5 | 11 | 5(5)/11 | 6.39E−03 | −1.38 |
| NP_0000230.1 | 4 | 4 | 17 | 4(4)/17 | 5.41E−03 | −1.38 |
| NP_0000164.4 | 2 | 2 | 15 | 2(2)/15 | 2.83E−02 | −8.05 |
| NP_0005555.2 | 2 | 2 | 7 | 2(2)/7 | 5.92E−03 | −7.56 |
| NP_0005013.1 | 2 | 2 | 9 | 2(2)/9 | 4.09E−02 | −7.99 |
| NP_0002695.1 | 3 | 3 | 27 | 3(3)/27 | 1.61E−02 | −7.56 |
| NP_0001104026.1 | 4 | 4 | 30 | 4(4)/30 | 7.73E−03 | −3.22 |
| NP_0002655.2 | 2 | 2 | 8 | 2(2)/8 | 2.21E−03 | −5.70 |
| NP_0000933.1 | 2 | 2 | 2 | 2(2)/2 | 3.95E−03 | −1.88 |
| NP_0001129174.1 | 1 | 1 | 7 | 1(1)/7 | 4.55E−02 | −0.84 |
| NP_0001034438.1 | 1 | 1 | 10 | 1(1)/10 | 2.28E−02 | −7.73 |
| NP_0004985.2 | 1 | 1 | 8 | 1(1)/8 | 4.96E−02 | −1.25 |
| NP_0000588.2 | 1 | 1 | 8 | 1(1)/8 | 1.92E−02 | −8.90 |
| NP_000558.2 | 1 | 1 | 3 | 1(1)/3 | 2.56E−02 | −0.30 |
| NP_004336.2 | 1 | 1 | 2 | 1(1)/2 | 3.38E−02 | −3.30 |
| NP_005208.1 | 1 | 1 | 5 | 1(1)/5 | 3.83E−02 | −4.90 |
| NP_002956.1 | 1 | 1 | 7 | 1(1)/7 | 1.54E−02 | −2.47 |
| NP_001138632.1 | 1 | 1 | 8 | 1(1)/8 | 1.00E−02 | −6.56 |
| NP_003290.1 | 1 | 1 | 4 | 1(1)/4 | 3.73E−02 | −1.30 |
| NP_057381.3 | 1 | 1 | 8 | 1(1)/8 | 3.73E−02 | −9.50 |
| NP_001919.2 | 3 | 2 | 15 | 3(2)/15 | 2.95E−02 | −5.09 |
| NP_002635.2 | 1 | 1 | 3 | 1(1)/3 | 4.89E−02 | −0.34 |
| NP_002584.2 | 1 | 1 | 6 | 1(1)/6 | 2.12E−02 | −2.59 |
| NP_055140.1 | 1 | 1 | 4 | 1(1)/4 | 2.98E−02 | −8.50 |
| P00748.2 | 1 | 1 | 1 | 1(1)/1 | 9.18E−03 | −5.41 |

### Example 3

### Detection of Axoglial-Apparatus Auto-Antigens in the CSF

In addition to their insulating function in the CNS, oligodendrocytes also trigger domain formation on axonal membranes, resulting in the nodal, paranodal and juxtaparanodal specializations of the axonal membrane. These axonal specializations together with the overlying and attached glial paranodal loops are known as the axoglial-apparatus (Pedraza, L., Huang, J. K. & Colman, DR., Neuron 30, 335-344 (2001); Huang, J. K., et al. Science 310, 1813-1817 (2005)).

Very little is known about the formation and assembly of the axoglial-apparatus in the CNS, however several key molecules exclusively localized to these areas have been identified. Table 5 summarizes known axoglial-apparatus proteins that are known to mediate axon-glia interaction in maintaining the integrity of the functional node of Ranvier. Table 5 also summarizes various molecules that mediate axon-glia interaction along the internode and which might indirectly play a role in maintaining the integrity of nodal and paranodal regions.

TABLE 5
Identified axoglial-apparatus proteins.
Known Axoglial-apparatus components

| | |
|---|---|
| NP_068367.1 | ADAM metallopeptidase domain 22 |
| Q96GW7.2 | Brevican core protein precursor |
| NP_054860.1 | cell recognition molecule Caspr2 precursor |
| NP_001834.2 | contactin 1 isoform 1 precursor |
| NP_778203.1 | contactin 1 isoform 2 precursor |
| Q02246.1 | Contactin-2 precursor (Axonin-1) (Axonal glycoprotein TAG-1) (TAX-1) |
| NP_861454.2 | Gliomedin [Homo sapiens] |
| P23515.2 | Oligodendrocyte-myelin glycoprotein precursor |
| NP_055905.2 | neurofascin precursor |
| Q92823.2 | Neuronal cell adhesion molecule precursor (Nr-CAM) (Ng-CAM-related) |
| P43146.1 | Netrin receptor DCC precursor |
| Q92752.2 | Tenascin-R precursor (TN-R) (Restrictin) (Janusin) |
| P13611.3 | Versican core protein precursor (Large fibroblast proteoglycan) |
| | (Chondroitin sulfate proteoglycan core protein 2) |
| NP_001119808.1 | versican isoform 2 |
| NP_004384.2 | dystroglycan 1 preproprotein |
| Q6ZMI3.1 | Gliomedin |

Other relevant components that mediate neuron-to-glia adhesion

| | |
|---|---|
| Q8IWZ3.1 | Ankyrin repeat and KH domain-containing protein 1 (Multiple ankyrin |
| | repeats single KH domain) (hMASK) |
| NP_647538.1 | attractin isoform 2 [Homo sapiens] |
| P80723.2 | Brain acid soluble protein 1 (BASP1 protein) (Neuronal axonal membrane |
| | protein NAP-22) (22 kDa neuronal tissue-enriched acidic protein) |
| Q13740.2 | CD166 antigen precursor (Activated leukocyte cell adhesion molecule) |
| Q9BY67.2 | Cell adhesion molecule 1 precursor (Immunoglobulin superfamily member |
| | 4) (Nectin-like protein 2) (NECL-2) (Tumor suppressor in lung cancer 1) |
| | (TSLC-1) (Synaptic cell adhesion molecule) |
| Q8N3J6.1 | Cell adhesion molecule 2 precursor (Immunoglobulin superfamily member |
| | 4D) (Nectin-like protein 3) |
| NP_001120645.1 | cell adhesion molecule 3 isoform 2 [Homo sapiens] |
| Q8N126.1 | Cell adhesion molecule 3 precursor (Immunoglobulin superfamily member |
| | 4B) (Nectin-like protein 1) (TSLC1-like protein 1) (Synaptic cell adhesion |
| | molecule 3) (Brain immunoglobulin receptor) |
| NP_660339.1 | cell adhesion molecule 4 [Homo sapiens] |
| Q8NFZ8.1 | Cell adhesion molecule 4 precursor (Immunoglobulin superfamily member |
| | 4C) (Nectin-like protein 4) (TSLC1-like protein 2) |
| NP_006605.2 | cell adhesion molecule with homology to L1CAM precursor [Homo |
| | sapiens] |
| NP_620481.2 | cell recognition protein CASPR4 isoform 2 [Homo sapiens] |
| NP_065923.1 | contactin 3 [Homo sapiens] |
| NP_783200.1 | contactin 4 isoform a precursor [Homo sapiens] |
| Q9P232.2 | Contactin-3 precursor (Brain-derived immunoglobulin superfamily protein |
| | 1) (BIG-1) (Plasmacytoma-associated neuronal glycoprotein) |
| Q9UQ52.1 | Contactin-6 precursor (Neural recognition molecule NB-3) (hNB-3) |
| P54764.1 | Ephrin type-A receptor 4 precursor (Tyrosine-protein kinase receptor SEK) |
| | (Receptor protein-tyrosine kinase HEK8) (Tyrosine-protein kinase TYRO1) |
| P54762.1 | Ephrin type-B receptor 1 precursor (Tyrosine-protein kinase receptor EPH- |
| | 2) (NET) (HEK6) (ELK) |
| P52799.1 | Ephrin-B2 precursor (EPH-related receptor tyrosine kinase ligand 5) |
| | (LERK-5) (HTK ligand) (HTK-L) |
| Q969P0.1 | Immunoglobulin superfamily member 8 precursor (CD81 partner 3) (Glu- |
| | Trp-Ile EWI motif-containing protein 2) (EWI-2) |
| NP_001091987.1 | immunoglobulin superfamily, member 4D isoform 2 [Homo sapiens] |
| NP_076493.1 | L1 cell adhesion molecule isoform 2 precursor [Homo sapiens] |
| Q96FE5.2 | Leucine-rich repeat and immunoglobulin-like domain-containing nogo |
| | receptor-interacting protein 1 precursor (Leucine-rich repeat neuronal |
| | protein 6A) |
| NP_996536.2 | myelin oligodendrocyte glycoprotein isoform beta4 precursor [Homo |
| | sapiens] |
| P43146.1 | Netrin receptor DCC precursor (Tumor suppressor protein DCC) |
| NP_851996.2 | neural cell adhesion molecule 1 isoform 2 [Homo sapiens] |
| P13591.2 | Neural cell adhesion molecule 1, 140 kDa isoform precursor (N-CAM 140) |
| NP_004531.2 | neural cell adhesion molecule 2 precursor [Homo sapiens] |
| O00533.2 | Neural cell adhesion molecule L1-like protein precursor (Close homolog of |
| | L1) |
| NP_001098720.1 | neurexin 3 isoform 3 precursor [Homo sapiens] |
| Q9ULB1.1 | Neurexin-1-alpha precursor (Neurexin I-alpha) |
| Q9P2S2.1 | Neurexin-2-alpha precursor (Neurexin II-alpha) |
| Q9Y4C0.3 | Neurexin-3-alpha precursor (Neurexin III-alpha) |
| NP_005001.3 | neuronal cell adhesion molecule isoform B precursor [Homo sapiens] |
| NP_148937.1 | platelet-derived growth factor beta isoform 2, preproprotein [Homo sapiens] |
| P23471.3 | Receptor-type tyrosine-protein phosphatase zeta precursor (R-PTP-zeta) (R- |
| | PTP-zeta-2) |

Several key molecules required for proper functioning of the axoglial-apparatus were identified in the CSF in our samples. For example, the molecules identified in the CSF include the axo-glial apparatus proteins Neurofascin, Contactin-2/TAG-1, Brevican, CASPR, Gliomedin, NrCAM, OMgp, Tenascin R, Contactin-1/F3, Versican and several others (Tables 3, 4, 5 and 8; FIGS. 6 and 8). In recent studies auto-antibodies to axoglial-apparatus auto-antigens, such as neurofascin and contactin, have been reported (Derfuss, T., et al., Proc Natl Acad Sci USA 106, 8302-8307 (2009); Mathey, E. K., et al., J Exp Med 204, 2363-2372 (2007)). The molecules identified in our screen are localized to very sharp domains smaller than 5 micrometer in length, a size that is equivalent to 1-2% the size of the internode formed by the compact myelin. The internode itself can range from several hundreds of a micrometer to a millimeter in length. It is possible that several molecules identified in our screen act as auto-antigens, and that auto-antibodies to these molecules can be screened using various assays known in the art (O'Connor, K. C., et al., Nat Med 13, 211-217 (2007); Quintana, F. J., et al., Proc Natl Acad Sci USA 105, 18889-18894 (2008)).

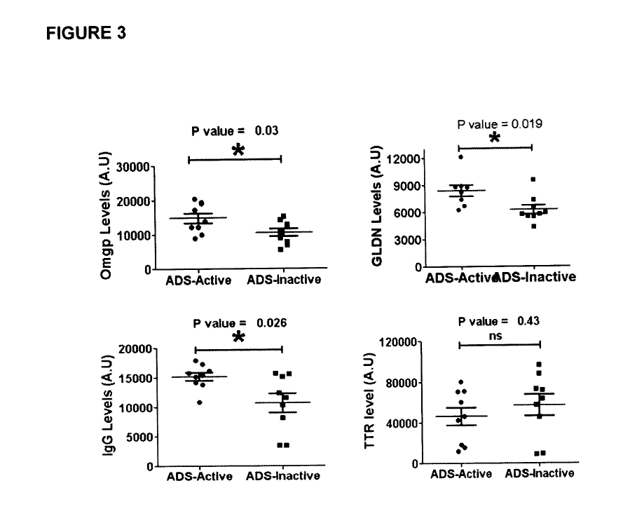

In addition, immunoblotting was performed on the CSF samples obtained during the first episode of CNS acute attack with antibodies directed against gliomedin and OMgp, axoglial-apparatus proteins that are proposed to cluster voltage gated sodium channels at the node of Ranvier. The immunoblotting experiments indicated elevated levels of these proteins in the CSF in the active cohort (FIG. 3), suggesting a greater extent of injury in subjects with elevated IgG levels. A known resident CSF molecule, TTR, showed no significant difference in concentration between the two cohorts.

The presence of axoglial-apparatus auto-antigens in the CSF samples suggests that the integrity and proper functioning of the axoglial-apparatus is compromised in the subjects. Our findings also suggest that nodal injury has occurred, as it is most likely that leakage of these molecules into the CSF is a result of an injury to the node of Ranvier.

In addition to molecules localized to the axoglial apparatus, several proteins that are localized to the outer or inner mesaxon as well as molecules known to mediate a strong axon-to-glia adhesion were also detected. These molecules include, for example, MOG, MAG, Necl-1, Necl-2, Necl-3, Necl-4, LGI-1, LGI-3, DCC, NCAM1, NCAM140, NCAM2, NCAM-L1-like, Neurexin1-4, Dystroglycan-1 and several others. The presence of these proteins in the CSF suggests an uncoupling of glial support to axons which might perturb neuron-to-glia adhesion.

In sum, in addition to confirming absence of well known auto-antigens PLP1 and MBP from CSF samples obtained from 24 children, we identified several key molecules that are required for the node of Ranvier and the paranodal assembly during an acute attack in juvenile patients. These proteins are known to be localized in the immediate vicinity of the node of Ranvier and are not participants in compact myelin. These data strongly suggest that the initial disruption of myelinated tracts that occurs during an acute attack in MS, at least in juvenile patients, is focused in the region of the node of Ranvier, and not, as is commonly thought, in the compact myelin sheath itself.

Importantly, the proteome signature obtained during the acute attack in MS can be accurately used to predict the progression from first CNS demyelination episode to MS. The results indicate that a quantitative analysis of axoglial-apparatus molecules either using multiple reaction monitoring or multiplexed ELISA assay can be used as a prognostic test for MS.

### Example 4

### CSF Proteome Signature During the First Acute Attack Accurately Predicts the Progression to MS

From the 23 children tested, eight experienced new disease activity over the follow-up period of 2 years, and were confirmed to develop MS using either of the following criteria: (1) developing, over time, one or more new clinical attacks (relapses), or (2) having evidence of new brain lesions developing over time by magnetic resonance imaging (MRI). Using this information, we determined whether the proteome signatures generated from the CSF collected during the first acute attack in these subjects could be used to predict the progression from CIS/ADS to MS. Using peptide profiling (Wang, W., et al., Anal Chem 75, 4818-4826 (2003)), 16,190 components were identified in the CSF, of which 7,367 components were linked to 3,839 peptide sequences corresponding to 1032 non-redundant proteins with an identification threshold of 0.7. Although many of these proteins (45%) were “singletons” (only one peptide per protein), which gives a lower degree of identification confidence, we identified 563 proteins with two or more unique peptides.

The modest cohort size tested means that the results are limited to relatively large effects. Nevertheless, we observed that roughly 7-10% of the proteins in the CSF show clear, statistically-significant differences in concentration in subjects who recovered compared to subjects who developed MS (Summarized in Table 6). These components were assigned to 67 non-redundant proteins that may be used to predict the progression of CIS/ADS to MS (Table 6).

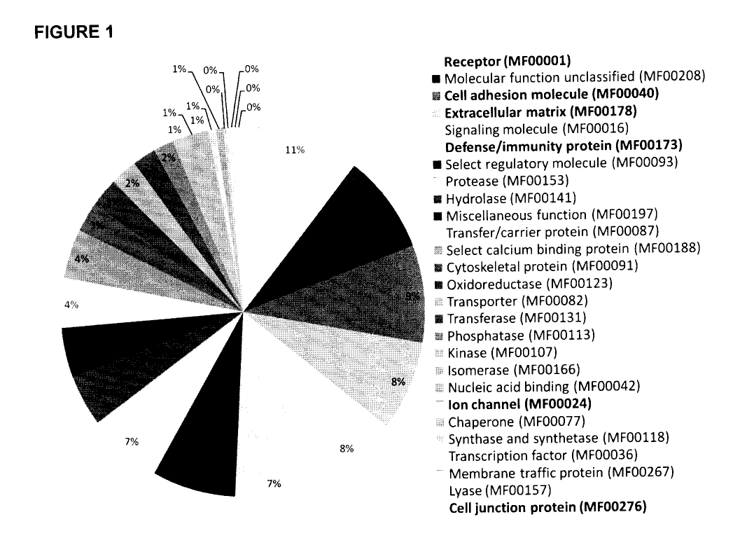

We then classified each protein according to molecular function as assigned by the online panther molecular classification freeware (available at http://www.pantherdb.org) (FIG. 1). From 563 proteins analyzed, 461 were assigned to 28 molecular function classes. It was determined that the most numerous proteins include cell adhesion, extracellular matrix, defense/immunity, and receptor proteins (8% or higher). Together with proteins of unknown function, these four classes make up more than half the identified proteins in both cohorts, suggesting an uncoupling of neuron-glia adhesion and an involvement of immunological response at an early stage of CNS inflammation (FIG. 1).

TABLE 6
Components showing statistically significant concentration difference
in CSF between the two cohorts (Active and Inactive).

| Total components | 16190 |
|---|---|
| P < 0.05 | 561 |
| P < 0.01 | 74 |
| P < .005 | 35 |
| P < 0.0001 | 4 |

| | Fold | trend | signif | trend | total | | | <ID |
|---|---|---|---|---|---|---|---|---|
| Accession # | Change | seq | comp. | comp. | comp. | Summary | Pmin | Prob.> |
| NP_620481.2 | 1.73 | 1 | 1 | 1 | 10 | 1(1)/10 | 2.24E−02 | −6.55 |
| NP_001001329.1 | 1.63 | 1 | 1 | 1 | 7 | 1(1)/7 | 2.38E−03 | −4.72 |
| NP_774959.1 | 1.61 | 6 | 6 | 6 | 41 | 6(6)/41 | 8.54E−04 | −3.85 |
| NP_001747.2 | 1.59 | 1 | 1 | 1 | 6 | 1(1)/6 | 2.26E−02 | −3.46 |
| NP_003005.2 | 1.57 | 1 | 1 | 1 | 2 | 1(1)/2 | 2.18E−02 | −2.72 |
| NP_001106283.1 | 1.55 | 1 | 1 | 1 | 5 | 1(1)/5 | 2.15E−02 | −9.10 |
| NP_001864.1 | 1.53 | 4 | 4 | 4 | 21 | 4(4)/21 | 4.79E−03 | −9.70 |
| NP_003276.3 | 1.53 | 1 | 1 | 1 | 3 | 1(1)/3 | 2.95E−02 | −4.93 |
| NP_683880.1 | 1.48 | 1 | 1 | 1 | 5 | 1(1)/5 | 1.81E−02 | −4.60 |
| P01859.2 | 1.44 | 1 | 1 | 1 | 3 | 1(1)/3 | 1.81E−02 | −5.62 |
| NP_001034438.1 | 1.37 | 4 | 5 | 5 | 31 | 5(5)/31 | 2.54E−03 | −6.62 |
| NP_000382.3 | 1.37 | 1 | 1 | 1 | 7 | 1(1)/7 | 9.75E−03 | −8.80 |
| NP_037511.2 | 1.35 | 1 | 1 | 1 | 3 | 1(1)/3 | 3.04E−02 | −4.25 |
| NP_001401.2 | 1.35 | 2 | 2 | 2 | 20 | 2(2)/20 | 2.33E−02 | −5.17 |
| NP_001839.2 | 1.33 | 8 | 9 | 9 | 20 | 9(9)/20 | 1.55E−03 | −5.15 |
| NP_067042.1 | 1.31 | 1 | 1 | 1 | 3 | 1(1)/3 | 7.12E−03 | −6.41 |
| NP_000304.2 | 1.26 | 5 | 6 | 6 | 25 | 6(6)/25 | 8.25E−03 | −7.15 |
| NP_705872.1 | 1.25 | 1 | 1 | 1 | 1 | 1(1)/1 | 2.71E−02 | −0.91 |
| NP_001073926.1 | 1.23 | 1 | 1 | 1 | 9 | 1(1)/9 | 4.31E−02 | −3.98 |
| NP_000051.1 | 1.23 | 5 | 5 | 5 | 13 | 5(5)/13 | 1.26E−02 | −8.76 |
| NP_006477.2 | 1.22 | 3 | 5 | 4 | 17 | 5(4)/17 | 7.74E−03 | −6.17 |
| NP_001001391.1 | 1.22 | 1 | 1 | 1 | 6 | 1(1)/6 | 2.93E−02 | −1.51 |
| NP_001013049.1 | 1.18 | 1 | 1 | 1 | 5 | 1(1)/5 | 3.80E−02 | −0.29 |
| NP_002406.1 | 1.16 | 2 | 3 | 2 | 3 | 3(2)/3 | 1.18E−03 | −6.38 |
| NP_699201.2 | 1.16 | 1 | 1 | 1 | 4 | 1(1)/4 | 4.70E−02 | −4.25 |
| NP_055267.1 | −1.12 | 1 | 1 | 1 | 8 | 1(1)/8 | 3.96E−02 | −1.32 |
| NP_056366.1 | −1.17 | 1 | 1 | 1 | 3 | 1(1)/3 | 4.31E−02 | −3.89 |
| NP_055851.1 | −1.20 | 3 | 3 | 3 | 16 | 3(3)/16 | 2.70E−02 | −3.98 |
| NP_003864.4 | −1.21 | 1 | 1 | 1 | 2 | 1(1)/2 | 2.57E−02 | −6.64 |
| NP_569711.2 | −1.23 | 2 | 2 | 2 | 11 | 2(2)/11 | 4.85E−02 | −5.01 |
| NP_009016.1 | −1.24 | 2 | 2 | 2 | 8 | 2(2)/8 | 2.86E−02 | −4.59 |
| NP_006735.2 | −1.24 | 3 | 3 | 3 | 21 | 3(3)/21 | 3.32E−02 | −1.71 |
| NP_002070.1 | −1.25 | 4 | 4 | 4 | 18 | 4(4)/18 | 2.72E−02 | −6.24 |
| NP_009212.1 | −1.27 | 1 | 2 | 2 | 4 | 2(2)/4 | 4.17E−02 | −2.04 |
| NP_002514.1 | −1.29 | 3 | 3 | 3 | 14 | 3(3)/14 | 9.29E−03 | −1.77 |
| NP_005336.3 | −1.31 | 3 | 3 | 3 | 7 | 3(3)/7 | 6.10E−03 | −3.45 |
| NP_002291.1 | −1.32 | 2 | 2 | 2 | 10 | 2(2)/10 | 1.02E−02 | −4.28 |
| NP_001128711.1 | −1.34 | 1 | 1 | 1 | 1 | 1(1)/1 | 2.26E−02 | −11.00 |
| NP_001543.2 | −1.35 | 2 | 2 | 2 | 13 | 2(2)/13 | 2.89E−02 | −6.87 |
| NP_476508.2 | −1.38 | 1 | 1 | 1 | 9 | 1(1)/9 | 2.16E−02 | −1.45 |
| NP_001327.2 | −1.38 | 1 | 1 | 1 | 3 | 1(1)/3 | 2.37E−02 | −4.93 |
| NP_570858.2 | −1.39 | 2 | 2 | 2 | 7 | 2(2)/7 | 1.69E−02 | −4.39 |
| NP_570925.2 | −1.42 | 1 | 1 | 1 | 8 | 1(1)/8 | 1.51E−02 | −6.41 |
| NP_001892.1 | −1.43 | 1 | 1 | 1 | 4 | 1(1)/4 | 2.88E−02 | −7.91 |
| NP_612457.1 | −1.43 | 1 | 1 | 1 | 2 | 1(1)/2 | 4.67E−02 | −8.50 |
| NP_000430.3 | −1.44 | 1 | 1 | 1 | 5 | 1(1)/5 | 1.87E−02 | −0.88 |
| NP_002558.1 | −1.45 | 2 | 2 | 2 | 19 | 2(2)/19 | 7.28E−03 | −4.75 |
| NP_005548.2 | −1.48 | 1 | 1 | 1 | 2 | 1(1)/2 | 2.27E−02 | −1.18 |
| NP_064575.1 | −1.48 | 1 | 1 | 1 | 4 | 1(1)/4 | 3.22E−02 | −3.75 |
| NP_001129127.1 | −1.50 | 1 | 1 | 1 | 3 | 1(1)/3 | 4.47E−02 | −4.93 |
| NP_006395.2 | −1.52 | 1 | 1 | 1 | 3 | 1(1)/3 | 1.69E−02 | −4.92 |
| NP_002347.5 | −1.53 | 2 | 2 | 2 | 4 | 2(2)/4 | 2.92E−03 | −4.79 |
| NP_001030024.1 | −1.54 | 1 | 1 | 1 | 4 | 1(1)/4 | 2.04E−03 | −2.21 |
| NP_001121089.1 | −1.54 | 7 | 7 | 7 | 20 | 7(7)/20 | 1.13E−02 | −6.15 |
| NP_001135749.1 | −1.55 | 1 | 1 | 1 | 1 | 1(1)/1 | 1.73E−02 | −4.68 |
| NP_872271.1 | −1.57 | 2 | 3 | 2 | 30 | 3(2)/30 | 2.52E−02 | −9.50 |
| NP_006308.3 | −1.63 | 7 | 8 | 8 | 10 | 8(8)/10 | 2.58E−03 | −5.39 |
| NP_001073332.1 | −1.67 | 1 | 1 | 1 | 3 | 1(1)/3 | 1.65E−02 | −1.12 |
| NP_002429.1 | −1.70 | 1 | 1 | 1 | 2 | 1(1)/2 | 3.87E−02 | −4.26 |
| NP_066953.1 | −1.72 | 1 | 1 | 1 | 1 | 1(1)/1 | 2.87E−02 | −4.26 |
| NP_068368.2 | −1.79 | 1 | 2 | 2 | 10 | 2(2)/10 | 1.18E−02 | −0.23 |
| NP_000474.2 | −1.79 | 1 | 1 | 1 | 4 | 1(1)/4 | 2.30E−02 | −2.70 |
| NP_940998.1 | −1.80 | 1 | 1 | 1 | 1 | 1(1)/1 | 2.74E−02 | −4.51 |
| NP_001123536.1 | −1.80 | 3 | 3 | 3 | 4 | 3(3)/4 | 1.49E−02 | −4.72 |
| NP_003245.1 | −1.83 | 2 | 2 | 2 | 11 | 2(2)/11 | 1.17E−02 | −9.15 |
| NP_981961.1 | −2.79 | 3 | 3 | 3 | 11 | 3(3)/11 | 2.30E−02 | −2.92 |
| NP_741960.1 | −2.96 | 1 | 1 | 1 | 1 | 1(1)/1 | 3.73E−02 | −2.47 |

### Example 5

### Axoglial Apparatus Proteins Detected in Pediatric ADS CSF, Their Expression in Isolated Fractions from Human CNS, and a Model of the Human Axoglial Apparatus

To extend the prior work in rodents and confirm that proposed axoglial apparatus molecules are indeed components of the human axoglial apparatus (and are not present in the compact myelin), myelin and axoglial apparatus fractions were isolated from freshly dissected human CNS tissue, using techniques previously described (Dhaunchak et al., Glia, 58: 1949-1960, 2010) and immunoblotted for novel (GLIOMEDIN, CASPR4) and known axoglial components from rodent studies (Susuki and Rasband, Curr. Opin. Cell Biol. 20: 616-623, 2008) (FIG. 6B). Mutually exclusive protein representations were found in the myelin and axoglial apparatus fractions isolated from the human tissue (FIG. 6B). A schematic of the human axoglial apparatus is shown in FIG. 6D.

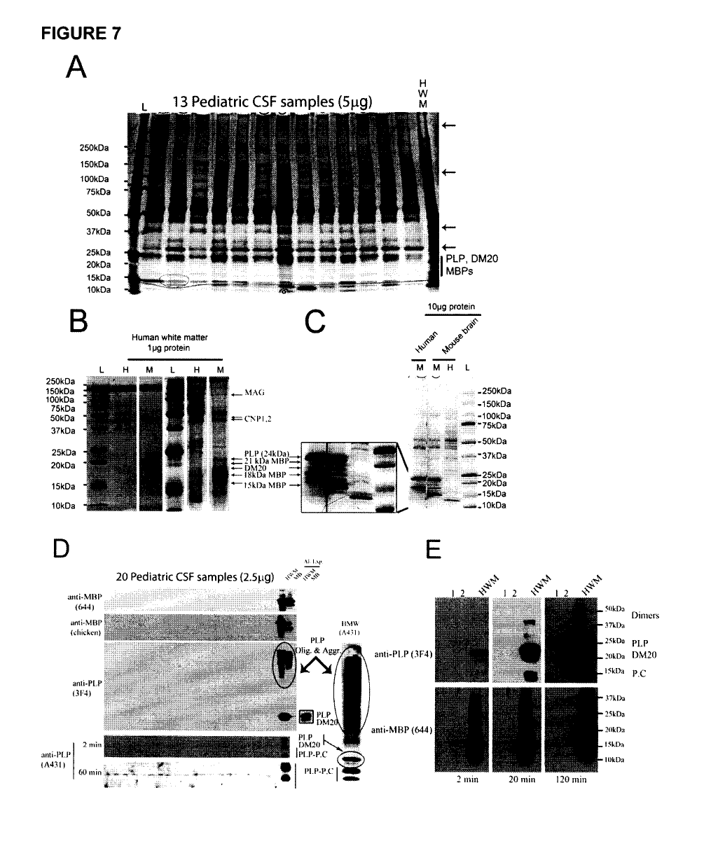

FIG. 6 shows axoglial apparatus proteins detected in pediatric ADS CSF, their expression in isolated fractions from human CNS, and a model of the human axoglial apparatus. FIG. 6A shows a summary of previously known axoglial apparatus and compact myelin proteins identified by mass spectroscopy and immunoblotting of CSF samples obtained from children presenting with an initial episode of acquired demyelinating syndrome (ADS). None of the compact myelin proteins were detected, despite these classical myelin proteins representing about 90% of the myelin protein content (FIG. 7). In contrast, from the known axoglial apparatus molecules, 65% of proteins expressed in this region were readily detected.

In FIG. 6B it is shown that human compact myelin and axoglial apparatus show reciprocal protein expression in purified CNS fractions. Known components (from rodent as well as human studies) were probed for their presence in isolated human myelin (M) or axoglial apparatus (Ax) fractions obtained from fresh CNS tissue, either resolved under reducing (black border) or non-reducing conditions (red border). Classical compact myelin (PLP, MBP) or axoglial apparatus specific (NEUROFASCIN, OMGP, VERSICAN) proteins showed reciprocal expression in respective fractions. However, non-compact myelin proteins (CNP), periaxonal membrane and mesaxonal proteins (MAG, MOG and NCAM) were detected in both fractions, as would be expected given their tight association to the axolemma. Due to their enrichment in axoglial fractions, several novel isoforms for known axoglial apparatus components were readily detected (data not shown). We also show for the first time that CASPR4 and GLIOMEDIN are components of CNS axoglial apparatus in humans.

FIG. 6C shows representative pediatric ADS CSF samples immunoprobed for presence of key axoglial apparatus proteins, i.e, NEUROFASCIN, OMGP and GLIOMEDIN. All three proteins were readily detected. In FIG. 6D is shown the complement of human axoglial apparatus formed by the paranodal loops of oligodendrocytes (OL), the axonal node of Ranvier, the paranode and the juxta-paranode. Proteins characteristic for each domain were either validated (see FIG. 6B) or are assembled based on a review of axoglial apparatus formation (Susuki and Rasband, Curr. Opin. Cell Biol. 20: 616-623, 2008).

### Example 6

### Absence of the Compact Myelin Auto-Antigens in the CSF

CSF samples obtained from a representative subset of children (n=13) were resolved under reducing and denaturing conditions on pre-cast gradient gels (4-20%) and visualized with silver staining (FIG. 7). Proteins corresponding to the molecular weights of PLP, DM20 or MBP were missing in the CSF of these children. The proteins are labeled next to their expected molecular weights in the lane loaded with human white matter homogenate (HWM). To confirm that the low molecular weight bands correspond to PLP, DM20 and MBP, myelin was purified from HWM tissue and 1 μg (FIG. 7B) or 10 μg (FIG. 7C) protein was resolved on high percentage gels.

PLP and MBP are major proteins of myelin and are readily detected with coomassie brilliant blue (CBB) or silver staining even when only 1 μg protein is resolved (FIG. 7B). CNP1,2 (non-compact myelin protein) and MAG (periaxonal membrane and mesaxon protein) are only visible with silver staining.

When 10 μg protein is resolved (FIG. 7C), even the less abundant CNP1.2 and MAG are readily detected with CBB staining. The major isoforms of human and murine MBP and PLP present in myelin are highlighted in FIG. 7C. In humans, the 18.5 kDa isoform of MBP is the major isoform while in mouse myelin the 15 kDa isoform predominates.

FIG. 7D shows that myelin proteins PLP, MBP and their isoforms are not detected in the CSF, when immunoprobed with two different antibodies recognizing two different epitopes. 2.5 μg protein was resolved for CSF samples and 300 ng for HWM. Only upon very long exposure with the polyclonal rabbit antibody against PLP (A431) was a weak band, slightly higher than the smallest proteolytic cleavage product of PLP (PLP-P.C; ˜8 kDa), detected (FIG. 7A). Such products are not detected with mouse monoclonal antibody (FIG. 7E).

CSF samples from two children (lane 1: ADS active, lane 2: ADS inactive) and HWM were immunoblotted for PLP (monoclonal 3F4 antibody) and MBP (polyclonal affinity purified 644 antibody) (FIG. 7E). Even after a very long exposure of up to 120 minutes neither the full length nor the proteolytic cleavage products of PLP and MBP were detected in the CSF.

### Example 7

### CSF Proteome of Children Presenting with ADS is Different Between ADS-MS and ADS-Monophasic Cohorts

CSF samples collected from 18 children presenting with an initial acquired demyelinating syndrome (ADS) were subjected to immunoblotting (FIG. 8A). In prospective follow up, 9 of the 18 children were ascertained to develop new MS-defining disease activity (ADS-MS; designated with ‘1’ in figure), while 9 exhibited no further disease activity (ADS-Monophasic; designated with ‘2’ in figure). One sample was loaded twice for quality control, hence 19 lanes are shown. HWM2 designates longer exposures of lanes loaded with HWM (human white matter). MB (mouse brain) lysates are shown on the right.

Densitometry compares levels of particular proteins in CSF of children subsequently diagnosed with MS (ADS-MS) vs children who remained monophasic (ADS-monophasic) (FIG. 8B). Consistent with prior reports, levels of CSF immunoglobulins (˜55 kDa) were significantly higher in ADS-MS children. This did not merely reflect overall increased levels of CSF proteins, as the ADS-MS and ADS-Monophasic cohorts have similar levels of the CSF-resident protein TTR (transthyretin; ˜14 kDa; the carrier of thyroid hormone and thyroxine). Levels of the axoglial proteins OMGP (˜60 kDa) and GLIOMEDIN (˜60 kDa), two molecules proposed to mediate clustering of the voltage gated sodium channels at the nodes of Ranvier, were also higher in the CSF of ADS-MS children. Levels of another key axoglial apparatus molecule, NEUROFASCIN, were not statistically different. Several isoforms of MAG (˜50 kDa, ˜60 kDa, ˜80 kDa and ˜150 kDa), a periaxonal membrane and mesaxon protein that has been shown to transmit survival signal to neurons, were detected at levels that were no different between the cohorts.

The top eight upregulated (FIG. 8A), and downregulated (FIG. 8B) proteins in the CSF of ADS-MS (n=8) compared to ADS-Monophasic (n=11) cohorts are listed with the fold change indicated in FIGS. 8C and 8D. Three axoglial apparatus proteins (highlighted in bold) are among the proteins showing greatest fold changes in concentration, suggesting structurally perturbed nodes of Ranvier. The raw data of all identified components, including those detected at different concentrations in the two cohorts, are included in the tables above.

Pediatric CSF peptide profiling identified additional axoglial, axolemmal and inner and outer meso-axonal proteins (known to mediate strong axon-to-glia adhesion), though these molecules were not obviously different in concentration when comparing the ADS-MS and ADS-Monophasic cohorts. The axoglial apparatus proteins included: Brevican, Contactin-1/F3, Contactin-2/TAG-1, Caspr, Gliomedin, Neurofascin, NrCAM, OMgp, Tenascin R, Versican, LGI-1 (see FIGS. 6 and 8 and Tables 3, 4, 5 and 8); axolemmal proteins and oligodendroglial outer or inner mesaxonal molecules included: Lingo-1, Necl-1, Necl-2, Necl-3, Necl-4, MOG, MAG, DCC, NCAMs, L1, Neurexin1-4, Dystroglycan-1 (FIGS. 6 and 8 and Tables 3, 4, 5 and 8). The presence of these molecules suggests perturbed integrity of the node of Ranvier and neuron to glia adhesion, which may reflect an uncoupling of glial support to axons. In addition, classification of identified proteins according to their molecular function (assigned by panther database) identified in the pediatric CSF molecules mediating axon-to-glia interaction and immune system interaction (see FIG. 1).

This screen establishes the first atlas of peptides that can be easily identified in CSF, at least in children with ADS, and provides excellent candidates for quantitative proteomics, and for biomarkers.

TABLE 8
Selected list of axoglial apparatus components and molecules mediating
axo-glia interaction identified in the pediatric ADS CSF.

| | |
|---|---|
| Accession# | Protein Description |

Known axoglial apparatus components identified in the CSF

| | |
|---|---|
| NP_068368.2 | Adam22 |
| NP_068767.3 | Brevican |
| NP_054860.1 | Caspr2 |
| NP_001834.2 | contactin 1 isoform 1 |
| NP_778203.1 | contactin 1 isoform 2 |
| NP_005067.1 | contactin 2 |
| NP_005206.2 | deleted in colorectal carcinoma (DCC) |
| NP_861454.2 | Gliomedin |
| NP_002535.3 | Oligodendrocyte-myelin glycoprotein precursor (OMgp) |
| NP_055905.2 | Neurofascin |
| NP_001032209.1 | neuronal cell adhesion molecule isoform A precursor (NrCAM) |
| NP_003276.3 | Tenascin-R precursor (TN-R) (Restrictin) (Janusin) |
| NP_004376.2 | versican isoform 1 |
| NP_001119808.1 | versican isoform 2 |

Suspected axoglial apparatus components identified in the CSF

| | |
|---|---|
| NP_647538.1 | attractin isoform 2 |
| NP_001618.2 | activated leukocyte cell adhesion molecule (ALCAM) |
| NP_001120645.1 | Cell adhesion molecule 3 precursor (Immunoglobulin superfamily member 4B) (NECL1) |
| NP_660339.1 | Cell adhesion molecule 4 precursor (Immunoglobulin superfamily member 4C) (NECL4) |
| | (TSLC1-like protein 2) |
| NP_006605.2 | cell adhesion molecule with homology to L1CAM precursor |
| NP_620481.2 | cell recognition protein CASPR4 isoform 2 |
| NP_065923.1 | contactin 3 |
| NP_783200.1 | contactin 4 isoform a precursor |
| NP_004384.2 | dystroglycan 1 preproprotein |
| NP_004084.1 | ephrin B2 |
| NP_004429.1 | ephrin receptor EphA4 |
| NP_872272.2 | ephrin receptor EphA5 isoform b precursor |
| NP_694854.2 | immunoglobulin superfamily, member 4D (NECL3) |
| NP_001091987.1 | immunoglobulin superfamily, member 4D isoform 2 (NECL2) |
| NP_443100.1 | immunoglobulin superfamily, member 8 |
| NP_001137435.1 | L1 cell adhesion molecule isoform 3 precursor |
| NP_116197.4 | LINGO-1, Leucine-rich repeat and immunoglobulin-like domain-containing nogo receptor- |
| | interacting protein 1, leucine-rich repeat neuronal 6A |
| NP_996536.2 | myelin oligodendrocyte glycoprotein isoform beta4 precursor |
| NP_851996.2 | neural cell adhesion molecule 1 isoform 2 |
| NP_004531.2 | neural cell adhesion molecule 2 precursor |
| O00533.2 | Neural cell adhesion molecule L1-like protein precursor (Close homolog of L1) |
| NP_001098720.1 | neurexin 3 isoform 3 precursor |
| NP_001129131.1 | neurexin 1 isoform alpha2 precursor |
| NP_620060.1 | neurexin 2 isoform alpha-2 precursor |
| NP_620063.1 | neurexin 2 isoform beta precursor |
| NP_004787.2 | neurexin 3 isoform 1 precursor |
| NP_001098720.1 | neurexin 3 isoform 3 precursor |
| NP_570925.2 | protein tyrosine phosphatase, receptor type, sigma isoform 4 precursor |
| NP_002842.2 | protein tyrosine phosphatase, receptor-type, zeta1 precursor |
| NP_075380.1 | reticulon 4 receptor precursor |
| NP_848665.1 | reticulon 4 receptor-like 2 |

In sum, the proteomic analysis of the CSF and serum samples collected from active and inactive children who presented with CIS/ADS provides a significant number of differentially expressed proteins between the two cohorts. The lists in Tables 3, 4 and 6 and FIGS. 6 and 8 provide a condensed summary of proteins significantly changing in concentration, excluding proteins for which only one peptide was tracked (so called singletons). Some brief comments now follow; these comments are intended to be illustrative only and are not intended to be exhaustive.

For the one-dimensional CSF study, although the total number of components with statistically significant change (p<0.05) was less than 3%, consistent changing direction among multiple peptide entries of the same protein was observed (found in Tables 3 and 6).

For the two-dimensional serum study, the total number of components with statistically significant change (p<0.05) was about 6%. Like the CSF study, some proteins showed consistent concentration change but did not show sufficient numbers of components at p<0.05 relative to all components tracked to appear in Table 4. These cases probably reflect a clear trend but greater biological variability than for some of the proteins in Table 4.

Another similarity between the two studies is the modest extent to which protein levels were observed to change. In the CSF study, no change was more than two-fold for proteins listed in Tables 3 and 6 although some changes were very clear.

### Materials and Methods

Human CSF and serum samples, about 750 μL each for CSF and 250 μL each for serum, consisting of 24 subjects total from two cohorts (active and inactive) were analyzed. The cohort identities were blinded during the sample processing. The cohort identities were only released after the completion of data collection.

Samples were frozen and then maintained in a −80° C. freezer before processing. Prior to sample processing, samples from identical subjects were combined. Out of 24 CSF samples, three samples were used for preliminary testing. The outcome of the preliminary testing indicated that there was sufficient volume for a one-dimensional (1D) CSF proteomic study, but not for a 2D study. A total of 20 CSF samples, as well as 20 serum samples, were then selected for the proteomic analysis. The remaining samples were saved in the −80° C. freezer. The experimental run order for the samples was usually prepared using a block randomization strategy with one sample from each cohort or time point within a block (pairing), and randomized order within each block. The cohort identities were initially blinded. Quality control samples were used as a first and last sample. No abnormal appearance was observed in any of the samples.

Human CSF for the cohorts was subjected to mass spectrometric analysis for proteomic profiling (differential expression analysis), and associated identifications. Approximately 750 μL of human CSF was processed per sample. For each sample, the material was analyzed with a one-dimensional (1D) liquid-phase separation approach.

Human serum for the cohorts was subjected to mass spectrometric analysis for proteomic profiling (differential expression), and associated identifications. Approximately 150 μL of human plasma was processed per sample. For each sample, the material was analyzed with a two-dimensional (2D) liquid-phase separation approach, termed DeepLook™ analysis.

The two biofluids each had 14 abundant proteins substantially depleted by an antibody-based affinity resin to increase the effective dynamic range of the measurements. The antibody column was manufactured by Agilent Technologies, model MARS™ Hu-14; the proteins depleted were albumin, IgG, IgA, IgM, transferrin, fibrinogen, apolipoprotein A-I, apolopoprotein A-II, haptoglobin, alpha-1-antitrypsin, alpha-1-acid glycoprotein, alpha-2-macroglobulin, complement C3, and transthyretin.

The remaining proteins were denatured, disulfide bonds were reduced, and sulfhydryl groups were carboxymethylated prior to digestion by modified trypsin. During this process, low molecular weight molecules were excluded during a buffer exchange step with a 5-kDa cut-off filter.

For the CSF samples, the desalted tryptic peptides were dissolved with 50 μL of aqueous 0.1% formic acid.

For the serum samples, four fractions of peptides were obtained by off-line (fraction collected) strong-cation-exchange (SCX) chromatography. The desalted tryptic peptides were dissolved with 60 μL of aqueous 0.1% formic acid.

Profiling Label-Free Quantification Strategy with MassView™.

PPD Biomarker Discovery Sciences Inc. has developed a novel approach to quantification of LC-MS data, applicable to large numbers of peptides and metabolites for the purpose of differential expression measurements and identification of biomarkers (see Wang et al., “Quantification of Proteins and Metabolites by Mass Spectrometry without Isotopic Labeling or Spiked Standards,” Analytical Chemistry 75, 4818-4826 (2003), and related U.S. Pat. No. 6,835,927). Because isotopic labeling is not employed, this method is called label-free quantification.

In this method, the majority of the biomolecular components cannot be predicted at the time of the laboratory study, eliminating any possibility of prior investigation of relative sensitivity factors (RSFs). Furthermore, methods based on introducing a known amount of a chemically analogous extraneous substance as an internal standard (i.e. “spiking” of a standard reference material) are not practical or possible, whether the analog is chemically identical and isotope-labeled (the isotope dilution method) or based on chemical similarity. One cannot anticipate which components will be differentially expressed in a discovery-based screen of thousands of components, and the sheer number of possible molecules prohibits this approach in a discovery setting.

Thus, the differential quantification method used herein relies on changes in analyte signal intensities which directly reflect their concentrations in one sample relative to another. Samples are not mixed nor are the samples otherwise manipulated beyond that required for the LC-MS analysis itself. The sample preparation and LC-MS conditions are carefully controlled for optimal results, and frequent quality control samples are analyzed to assure stable, reproducible performance. There is no isotopic labeling of the analytes. Data from each sample is normalized relative to a reference file with a single normalization coefficient determined from the median of ratios of intensities of hundreds to thousands of components between the file in question and the reference file. Since it could not be predicted beforehand which protein components would be differentially expressed, methods based on introducing a known amount of a chemically analogous exogenous substance as an internal standard could not be used.

Identification of Peptides and Proteins.

Peptides of interest are linked by accurate mass and chromatographic retention time to separate tandem mass spectrometry (MS/MS) experiments, in this case using a linear ion-trap mass spectrometer (Thermo, model LTQ). The resulting MS/MS spectra contain fragmentation patterns with characteristic peptide backbone cleavages. Each MS/MS raw spectrum from an isolated precursor ion is compared with a human sequence database to find a match, and hence identification. The human database used is a nonredundant combination of RefSeq+Swissprot+PIR. A match-quality score is obtained.

To produce this match-quality score, Mascot™ software from Matrix Science Ltd. (London, UK) was used for the peptide identification software. This software is popular in the proteomics field, but has significant limitations especially when it comes to the grey zone between high and low confidence scores. In new informatics developments made by PPD Biomarker Discovery Sciences Inc., we have combined peptide identification match-quality scores with a prediction of reverse-phase chromatography peptide retention time which is compared with the observed retention time. This approach can also be applied to results from other search engines.

As a further innovation developed by PPD Biomarker Discovery Sciences Inc., a retention index, or RI, system is used, where the index adjusts the retention time to about 25 landmark chromatographic peaks. This system reduces uncertainties associated with any one specific LC-MS system used. It is noted that multiple profiling and tandem LC-MS systems can be used for any study. For the implementation herein, approximately 17 RI units equalled one minute of retention time. A 1-hour water/acetonitrile gradient was used for the on-line reverse-phase chromatography. This RI approach allows for more precise peptide and protein identifications by minimizing variations between instruments, as well as small changes over time on an instrument.

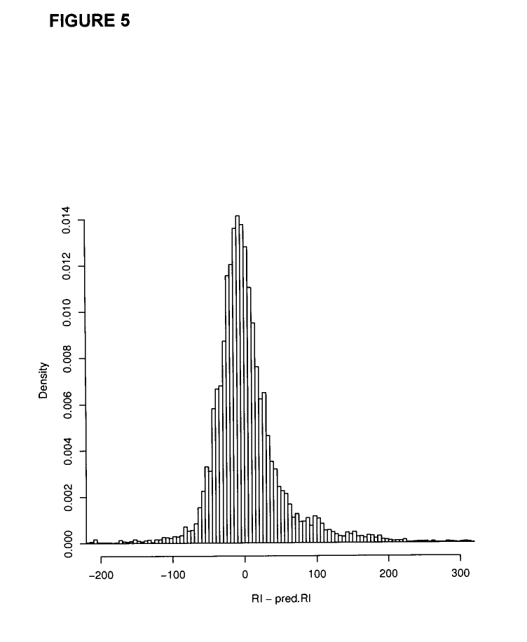

Absolute peptide identification probabilities were then computed by unsupervised machine learning in an expectation-maximization (EM) algorithm using the differences between observed and predicted retention time/index and Mascot scores. No distributional assumptions were made with regard to functional forms. The observed difference between predicted and observed retention index is found in FIG. 5. The retention time is predicted using amino acid composition throughout the peptide and specifically at the amino-terminus, as well as peptide length, following the approach by Krokhin et al, Mol. Cell. Proteomics 3, 908-19, 2004. However, this probability calculation is a new development by PPD's Biomarker Discovery Sciences Inc. group. In this study, a probability threshold of 0.7 was applied, with the computed probability reported for each peptide.

It is important to note that after this peptide identification probability is computed based on tandem mass spectra, there is a further constraining filter applied during linking the identification result to the profiling accurate mass and retention index through the use of narrow m/z and retention index uncertainty acceptance windows. A post-acquisition nonparametric mass calibration was performed as described by Becker et al, Analytical Chemistry 79, 1702-07 (2007).

Statistics.

We computed univariate statistics for each component, including a p-value from a comparison of cohort intensities. First a Shapiro-Wilk test for normality of the differences between cohorts for a given component was performed. Depending on whether the hypothesis of normality is rejected at the 0.05 level, we applied either a Student t-test or a Wilcoxon signed rank test to the within-pair differences, testing the hypothesis that the mean difference (or median, for Wilcoxon tests) is zero. In addition to p-values, we report several other statistics including fold change (computed as the median of cohort intensity ratios), absolute value of the effect size, and area under the receiver-operator characteristic curve (ROC_AUC). A glossary of terms and abbreviations is found in Table 7.

For those components that had predominantly missing values in one cohort but not in another, a p-value was computed, called p_count, by testing the hypothesis that the observed missing values for a given component were distributed randomly between the two groups compared, using Fisher's exact test. Low values of p_count indicate a greater degree of segregation of missing values into just one group than would be likely under pure randomization. In general, for each component, a minimum p-value was used between that calculated by t-test or Wilcoxon signed rank test (depending on normality—the great majority are normal) and the p_count value.

Paired statistical analyses were performed for this study. For computation of concentration fold change, the active cohort has its concentration in the numerator of the ratio.

TABLE 7
Glossary of terms.

| Legend/Abbreviation | Description |
|---|---|
| # | Serial number of molecule on Summary page |
| # Components | Number of molecular ions tracked whose expression ratios were used to calculate an |
| | average Exp. Ratio |
| # Peptides | Number of peptides from a given protein that show significant differential expression |
| <Exp. Ratio> | Mean of expression ratios from all contributing components |
| <p> | Mean P Value |
| 1D | One-Dimensional (chromatography) |
| 2D | Two-Dimensional (chromatography) |
| Accession # | Identification Number from NCBI's RefSeq Database |
| Ave. Peptide Score | Average numerical score from protein identification software matching raw data to |
| | NCBI database entries |
| Avg | Average; same as Arithmetic Mean |
| Chemical Name | Chemical Identification |
| Component | A molecular ion tracked and quantified for LC-MS (one molecular ion includes all of |
| | its isotopes); separately resolved chromatographic peak for GC-MS |
| Component # | Number used to denote a given component |
| Count | Number of subjects per study group with detected intensity for a specific component |
| CountDiff | Count difference between study groups; difference between two study groups of the |
| | number of subjects reporting an above-threshold intensity for a given component |
| CountDiffmin/max | Minimum/maximum number by which 2 groups must differ in count (appearance) to |
| | be categorized as a Count Diff |
| CV | Coefficient of variation |
| DeepLook ™ analysis | PPD's two-dimensional LC-MS analysis for proteomics |
| Detail Sheet | Mid-level view spreadsheet reporting changing molecules, for all entries |
| DM(mD) | Difference between observed mass and theoretical mass of matched peptide (in |
| | milliDaltons) |
| DM(ppm) | Difference between observed mass and theoretical mass of matched peptide (in parts |
| | per million) |
| Exp. Ratio | Expression ratio. Mean of the ratio of paired intensities, Group 1/Group 2 |
| Fold Change | Expression change factor; Positive indicates intensity increase, (negative indicates |
| | decrease). Group 1/Group 2 |
| gi # | Identification Number from NCBI's RefSeq Database |
| LC-MS | Liquid Chromatography-Mass Spectrometry |
| m/z | Mass-to-charge ratio; fundamental measure in mass spectrometry |
| M + H | Protonated parent mass |
| Max Mass | Maximum mass observed in a specific GC library entry |
| Max. Acc. Mass | The maximum accurately determined mass (GC-MS) |
| Mean | Arithmetic Mean |
| Median | The number in the middle of a set of numbers |
| Metab. | Metabolome |
| N Test | Binary result from test for normality (Shapiro-Wilk). 0: non-normal 1: normal |
| NA | Data point Not Available, below detection threshold |
| P_count | The probability that observed missing values for a given component are distributed |
| | randomly between the two groups compared, using Fisher's exact test |
| P Non Para | P value from nonparametric mean comparison test (Wilcoxon-Mann-Whitney test) |
| P Para | P value from parametric t-test for mean comparison |
| P used | P value assigned to a component resulting from the minimum value of the parametric |
| | or non-parametric test, depending on test for normality, compared with P_count |
| Peptide | Sequence of an identified peptide; All individually identified charge states for a given |
| | peptide are listed. |
| Peptide/Mass | Sequence of peptide identified or accurate mass of metabolite identified |
| Protein Description | Information on the protein identified, as contained in the NCBI queried database |
| R.T. | Chromatographic retention time |
| Raw Data Sheet | Most detailed spreadsheet reporting raw intensities for each subject for all |
| | components (molecules) |
| Score | Numerical confidence score used in peptide identification via matching to a database |
| SD | Standard Deviation |
| Summary Sheet | Top-level view spreadsheet reporting changing molecules, only for biologically |
| | identified molecules |
| Trend | “Up” for increased and “Down” for decreased expression level, Group 1 relative to |
| | Group 2 |
| T-Test | Two-sample t-test for mean comparison |
| z | Charge state of a molecular ion |

The contents of all documents and references cited herein are hereby incorporated by reference in their entirety.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. (canceled)

2. A method for diagnosing multiple sclerosis in a patient having pediatric-onset CNS demyelination, comprising the steps of determining the level of a biomarker in a fluid sample taken from the patient and in fluid samples taken from control subjects, wherein a change in concentration of the biomarker in the patient relative to concentration of the biomarker in the control subjects is diagnostic of multiple sclerosis.

3. A method for monitoring disease progression in a subject having multiple sclerosis (MS) or monitoring therapeutic efficacy of an anti-MS treatment, comprising the steps of isolating fluid samples from a subject at different time points and monitoring the level of a biomarker in the fluid samples taken from the subject, wherein an increase or decrease of the concentration of the biomarker in the samples over time indicates progression or regression of multiple sclerosis.

4. (canceled)

5. The method of claim 2, wherein the fluid sample is cerebrospinal fluid, blood or serum.

6. The method of claim 2, wherein elevation or diminution of the concentration of the biomarker in the subject indicates the subject is at high risk for developing multiple sclerosis.

7. (canceled)

8. The method of claim 2, wherein the biomarker is a nodal protein; a protein associated with the axoglial-apparatus in the CNS; a protein associated with cell adhesion, extracellular matrix or immunological response; a protein listed in Tables 3, 4 and/or 6; a protein listed in FIGS. 6 and/or 8; or one or more than one protein listed in Tables 3, 4 or 6 and/or FIG. 6 or 8.

9-11. (canceled)

12. The method of claim 2, wherein a combination of two or more biomarkers is used for diagnosis, prognosis or monitoring.

13. (canceled)

14. The method of claim 2, wherein the subject is a pediatric subject.

15-16. (canceled)

17. A method for predicting the likelihood of a subject with CIS/ADS developing multiple sclerosis (MS), comprising: (a) assaying the level of a protein in a first fluid sample obtained from the subject; and (b) comparing the level of the protein determined in (a) with the level of the protein in a second fluid sample obtained from a control subject; wherein a change in the level of the protein in the first fluid sample compared to the level of the protein in the second fluid sample indicates the likelihood of the subject developing MS.

18. The method of claim 17, wherein the control subject is: a subject who has CIS/ADS but does not develop MS; a subject who does not have CIS/ADS; a subject who does not have pediatric onset CNS demyelination; or a healthy subject.

19. (canceled)

20. The method of claim 17, wherein the protein is one or more of the proteins listed in Tables 3, 4 and 6 and/or FIGS. 6 and/or 8.

21. (canceled)

22. The method of claim 17, wherein the fluid sample is cerebrospinal fluid, blood or serum.

23. The method of claim 17, wherein the subject is a pediatric subject.

24-25. (canceled)

26. The method of claim 17, wherein the protein is one or more than one nodal protein or protein associated with the axoglial-apparatus in the CNS, or wherein the protein is one or more than one protein associated with cell adhesion, extracellular matrix or immunological response.

27-31. (canceled)

32. The method of claim 17, wherein the protein is reelin, fibulin-1, fibulin-3, collagen alpha-1 (VI), carboxypeptidase E, brain acid soluble protein 1 (neuronal axonal membrane protein), chromagranin A, macrophage colony-stimulating factor 1 receptor, fructose-bisphosphate aldolase A, or a combination thereof.

33. The method of claim 32, wherein elevation of the concentration of reelin, fibulin-1, fibulin-3, collagen alpha-1 (VI), or carboxypeptidase E, in the subject compared to control indicates that the subject has or is at risk of developing multiple sclerosis, or that the subject has active CIS/ADS; and/or wherein reduction of the concentration of brain acid soluble protein 1 (neuronal axonal membrane protein), chromagranin A, macrophage colony-stimulating factor 1 receptor, fructose-bisphosphate aldolase A indicates that the subject has or is at risk of developing multiple sclerosis, or that the subject has active CIS/ADS; wherein the bodily fluid is cerebrospinal fluid (CSF).

34-37. (canceled)

38. The method of claim 17, wherein the protein is alpha-1-B-glycoprotein, platelet glycoprotein Ib, platelet p47 protein (pleckstrin), platelet basic protein (C-X-C motif chemokine 7), antithrombin III, apolipoprotein A-I, attractin, carboxypeptidase N, complement components C1r and C7, hepatocyte growth factor activator, cytoplasmic actin 2, extracellular matrix protein 1, filamin A, neutrophil defensin 3, neutraphil gelatinase-associated lipocalin, SH3 domain-binding glutamic acid-rich-like protein 3, talin-1, thrombospondin-1, transgelin-2, tropomyosin 3, tropomyosin alpha-4, or a combination thereof.

39. The method of claim 38, wherein elevation of the concentration of alpha-1-B-glycoprotein, platelet motif chemokine 7), antithrombin III, apolipoprotein A-I, attractin, carboxypeptidase N, complement components C1r and C7, or hepatocyte growth factor activator in the subject compared to control indicates that the subject has or is at risk of developing multiple sclerosis, or that the subject has active CIS/ADS; and/or wherein reduction of the concentration of cytoplasmic actin 2, extracellular matrix protein 1, filamin A, neutrophil defensin 3, neutraphil gelatinase-associated lipocalin, SH3 domain-binding glutamic acid-rich-like protein 3, talin-1, thrombospondin-1, transgelin-2, tropomyosin 3, tropomyosin alpha-4 in the subject compared to control indicates that the subject has or is at risk of developing multiple sclerosis, or that the subject has active CIS/ADS; and the bodily fluid is serum.

40-43. (canceled)

44. The method of claim 17, wherein the protein comprises: a first protein which is Reelin, PKC substrate 80K-H, CASPR4, corticosteroid binding globulin precursor, secreted frizzled-related protein 4, glutamate receptor AMPA 4 isoform 3, carboxypeptidase E preprotein, Tenascin R, or a combination thereof; and/or a second protein which is CamKIIa, CD163 antigen isoform b, Tissue inhibitor of metalloproteinase 1, Growth associated protein 43 isoform 1, Sulfatase 2 isoform b precursor, Apolipoprotein C-II precursor, ADAM 22, Peptidylprolyl isomerase A, or a combination thereof; and wherein elevation of the concentration of the first protein in the CSF in the subject compared to control and/or reduction of the concentration of the second protein in the CSF in the subject compared to control indicates that the subject has or is at risk of developing multiple sclerosis, or that the subject has active CIS/ADS.
